SEQUENCE LISTING (17 sequences)

SEQ ID NO: 1 - DNA, 186 bp, Artificial
  Chemically Synthesized Oligonucleotide
gacaccaccgaggcccgccacccccacccccccgtgaccacccccaccaccaccgaccggaagggcaccaccgccgaggagctggccggcatcggcatcctgaccgtgatcctgggcggcaagcggaccaacaacagcacccccaccaagggcgaattctgcagatatccatcacactggcggccg

SEQ ID NO: 2 - protein, 61 aa, Artificial
  Chemically Synthesized polypeptide
DTTEARHPHPPVTTPTTDRKGTTAEELAGIGILTVILGGKRTNNSTPTKGEFCRYPSHWRP

SEQ ID NO: 3 - protein, 214 aa, Artificial
  Chemically Synthesized polypeptide
DIQMTQTTSSLSASLGDRVTISCRASQDISNYLNWYQQKPDGTVKLLIYYTSILQLGVPSRFSGSGSETDYSLTISNLEQEDIATYFCQQGDSLPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 447 aa, Artificial
  Chemically Synthesized polypeptide
QVTLKESGPGILQPSQTLSLTCSFSGFSLSTSGMSVGWIRQPSGKGLEWLAHIWWNDDKYYNPVLKSRLTISKETSNNQVFLKIASVVSADTATYYCARFYGNCLDYWGQGTTLTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAS

SEQ ID NO: 5 - protein, 781 aa, Artificial
  Chemically Synthesized Polypeptide
QVTLKESGPGILQPSQTLSLTCSFSGFSLSTSGMSVGWIRQPSGKGLEWLAHIWWNDDKYYNPVLKSRLTISKETSNNQVFLKIASVVSADTATYYCARFYGNCLDYWGQGTTLTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKASDTTEPATPTTPVTTDTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECLKYVRSAKLRMVHHHHHH

SEQ ID NO: 6 - protein, 781 aa, Artificial
  Chemically Synthesized Polypeptide
QVTLKESGPGILQPSQTLSLTCSFSGFSLSTSGMSVGWIRQPSGKGLEWLAHIWWNDDKYYNPVLKSRLTISKETSNNQVFLKIASVVSADTATYYCARFYGNCLDYWGQGTTLTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKASDTTEPATPTTPVTTDQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLAHHHHHH

SEQ ID NO: 7 - protein, 522 aa, Artificial
  Chemically Synthesized Polypeptide
QVTLKESGPGILQPSQTLSLTCSFSGFSLSTSGMSVGWIRQPSGKGLEWLAHIWWNDDKYYNPVLKSRLTISKETSNNQVFLKIASVVSADTATYYCARFYGNCLDYWGQGTTLTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKASNSPQNEVLYGDVNDDGKVNSTDLTLLKRYVLKAVSTLPSSKAEKNADVNRDGRVNSSDVTILSRYLIRVIEKLPI

SEQ ID NO: 8 - DNA, 36 bp, Artificial
  Chemically Synthesized Oligonucleotide
ggatggtgggaagatggatacagttggtgcagcatc

SEQ ID NO: 9 - DNA, 48 bp, Artificial
  Chemically Synthesized Oligonucleotide
ctaggaacagtcagcacgggacaaactcttctccacagtgtgaccttc

SEQ ID NO: 10 - DNA, 37 bp, Artificial
  Chemically Synthesized Oligonucleotide
gtcactggctcagggaaatagcccttgaccaggcatc

SEQ ID NO: 11 - DNA, 31 bp, Artificial
  Chemically Synthesized Oligonucleotide
ccaggcatcctagagtcaccgaggagccagt

SEQ ID NO: 12 - DNA, 38 bp, Artificial
  Chemically Synthesized Oligonucleotide
ggtgctggaggggacagtcactgagctgctcatagtgt

SEQ ID NO: 13 - DNA, 107 bp, Artificial
  Chemically Synthesized Oligonucleotide
ctagttgctggctaatggaccccaaaggctccctttcctggagaatacttctgtttctctccctggcttttgagttgtcgtacggattaattaagggcccactcgag

SEQ ID NO: 14 - DNA, 17 bp, Artificial
  Chemically Synthesized Oligonucleotide
gctagctgattaattaa

SEQ ID NO: 15 - DNA, 100 bp, Artificial
  Chemically Synthesized Oligonucleotide
ctagttgctggctaatggaccccaaaggctccctttcctggagaatacttctgtttctctccctggcttttgagttgtcgtacggattaattaagggccc

SEQ ID NO: 16 - DNA, 70 bp, Artificial
  Chemically Synthesized Oligonucleotide
gctagcgatacaacagaacctgcaacacctacaacacctgtaacaacaccgacaacaacacttctagcgc

SEQ ID NO: 17 - DNA, 119 bp, Artificial
  Chemically Synthesized Oligonucleotide
gctagccccattctgagccccctgaccaaaggcattctgggctttgtgtttaccctgaccgtgcccagcgaacgcaagggtatacttggattcgttttcacacttacttaagcggccgc

(19) United States
(12) Patent Application Publication
(10) Pub. No.: US 2008/0254047 A1
(43) Pub. Date: Oct. 16, 2008

(54) Activation of Human Antigen-Presenting Cells Through CLEC-6

(75) Inventors: Jacques F. Banchereau, Dallas, TX (US); SangKon Oh, Baltimore, MD (US); Gerard Zurawski, Midlothian, TX (US); Sandra Zurawski, Midlothian, TX (US); Dapeng Li, Dallas, TX (US)

Correspondence Address: CHALKER FLORES, LLP, 2711 LBJ FRWY, Suite 1036, DALLAS, TX, 75234, US

(73) Assignee: BAYLOR RESEARCH INSTITUTE, Dallas, TX (US)

(21) Appl. No.: 12/036,095

(22) Filed: Feb. 22, 2008

Related U.S. Application Data
(60) Provisional application No. 60/891,418, filed on Feb. 23, 2007.

(51) Int. Cl. A61K 39/00; C12N 5/02; C12N 5/16; C07K 16/18; A61P 37/04

(52) U.S. Cl. 424/184.1; 435/375; 435/377; 435/326; 530/387.1; 435/325

(57) ABSTRACT

The present invention includes compositions and methods for using novel anti-CLEC-6 antibodies and fragments thereof for modulating the activity of immune cells.

### CROSS-REFERENCE TO RELATED APPLICATIONS

This application claims priority to U.S. Provisional Application Ser. No. 60/891,418, filed Feb. 23, 2007 and is related to U.S. Provisional Application Serial No. __/___,___ filed Feb. 22, 2008, and Serial No. __/___,___, filed Feb. 22, 2008, the entire contents of which are incorporated herein by reference.

### STATEMENT OF FEDERALLY FUNDED RESEARCH

This invention was made with U.S. Government support under Contract No. 1U19AI057234-0100003 awarded by the NIH. The government has certain rights in this invention.

### TECHNICAL FIELD OF THE INVENTION

The present invention relates in general to the field of antigen presentation and immune cell activation, and more particularly, to the activation of immune cells through the CLEC-6 C-type lectin.

### BACKGROUND OF THE INVENTION

Without limiting the scope of the invention, its background is described in connection with dendritic cells.

Dendritic cells play a pivotal role in controlling the interface of innate and acquired immunity by providing soluble and intercellular signals, followed by recognition of pathogens. These functions of DCs are largely dependent on the expression of specialized surface receptors, ‘pattern recognition receptors’ (PRRs), represented, most notably, by toll-like receptors (TLRs) and C-type lectins or lectin-like receptors (LLRs) (1-3). In the current paradigm, a major role of TLRs is to alert DCs to produce interleukin 12 (IL-12) and other inflammatory cytokines for initiating immune responses. C-type LLRs operate as constituents of the powerful antigen capture and uptake mechanism of macrophages and DCs (1). Compared to TLRs, however, LLRs might have broader ranges of biological functions that include cell migrations (4), intercellular interactions (5). These multiple functions of LLRs might be due to the facts that LLRs, unlike TLRs, can recognize both self and nonself. However, the complexity of LLRs, including the redundancy of a number of LLRs expressed in immune cells, has been one of the major obstacles to understand the detailed functions of individual LLRs. In addition, natural ligands for most of these receptors remain unidentified. Nonetheless, evidence from recent studies suggests that LLRs, in collaboration with TLRs, may contribute to the activation of immune cells during microbial infections (6-14).

### SUMMARY OF THE INVENTION

The present invention includes compositions and methods for using anti-human CLEC-6 monoclonal antibodies (mAbs) and characterized their biological functions that are the basis of envisioned therapeutic applications of anti-CLEC-6 mAbs and their surrogates. The invention includes contacting antigen presenting cells, such as dendritic cells (DCs) that express CLEC-6, and that it plays a role in the uptake of antigens associated with particular DC activation that results in altered humoral and cellular immune responses. The inventors have developed and characterized unique agents capable of activating cells bearing CLEC-6, as well as the effect of the resulting changes in cells receiving these signals regards action on other cells in the immune system. These effects (either alone, or in concert with other signals (i.e., co-stimulation)) are highly predictive of therapeutic outcomes for certain disease states or for augmenting protective outcomes in the context of vaccination.

It was found that CLEC-6, one of the LLRs, is functional in terms of cell (including DC) activation by either alone or in collaboration with other cellular signals. CLEC-6-mediated cell activation was induced by anti-CLEC-6 mAbs, and therefore anti-human CLEC-6 mAbs or their surrogates will be useful for developing reagents against diseases.

The present invention includes compositions and methods for increasing the effectiveness of antigen presentation by a CLEC-6-expressing antigen presenting cell by contacting the antigen presenting cell with an anti-CLEC-6-specific antibody or fragment thereof, wherein the antigen presenting cell is activated. The antigen presenting cell may be an isolated dendritic cell, a peripheral blood mononuclear cell, a monocyte, a myeloid dendritic cell and combinations thereof. In one specific embodiment, the antigen presenting cell is an isolated dendritic cell, a peripheral blood mononuclear cell, a monocyte, a B cell, a myeloid dendritic cell and combinations thereof that have been cultured in vitro with GM-CSF and IL-4, interferon alpha, antigen and combinations thereof. The method may also include the step of activating the antigen presenting cells with GM-CSF and IL-4, wherein contact with the CLEC-6-specific antibody or fragment thereof increases the surface expression of CD86 and HLA-DR on the antigen presenting cell.

It has been found that the present invention can be used to activate antigen presenting cells with the CLEC-6-specific antibody or fragment thereof to increases the surface expression of CD86, CD80, and HLA-DR on the antigen presenting cell. If the antigen presenting cells are dendritic cells (DCs), DCs activated with the CLEC-6-specific antibody and GM-CSF and IL-4 to have the gene expression pattern of FIG. 4. The antigen presenting cells activated with a CLEC-6-specific antibody secrete IL-6, MIP- 1 a, MCP- 1, IP- 10, TNFa and combinations thereof, and if the APCs are dendritic cells, they secrete IL-6, MIP- 1 a, MCP-1, IP-10, TNFa, IL-12p40, IL- 1 a, IL- 1 b and combinations thereof. When activating dendritic cell that has been contacted with GM-CSF and IL-4 or Interferon alpha, the CLEC-6-specific antibody or fragment thereof and the CD40 ligand further increase the activation of the dendritic cells. When contacted with GM-CSF and IL-4 or Interferon alpha and the CLEC-6-specific antibody or fragment the DCs increased their co-stimulatory activity.

In another embodiment, the method of the present invention can be used to activate antigen presenting cells by co-activating the antigen presenting cell through the TLR9 receptor and the CLEC-6 lectin, wherein the cells increase cytokine and chemokine production, and even trigger B cells proliferation. It has also been found that co-activating antigen presenting cells with CLEC-6 and LOX-1 in the presence of B cells, induce the B cell immunoglobulin to class-switch. The TLR9 receptor may be activated with at least one of a TLR9 ligand, an anti-TLR9 antibody of fragments thereof, an anti-TLR9-anti-CLEC-6 hybrid antibody or fragment thereof, an anti-TLR9-anti-CLEC-6 ligand conjugate. Examples of the CLEC-6-specific antibody or fragment thereof may be selected from clone 12H7, 12E3, 9D5, 20H8 and combinations thereof. Dendritic cells activated through the CLEC-6-receptor with the CLEC-6-specific antibody or fragment thereof also activate monocytes, dendritic cells, peripheral blood mononuclear cells, B cells and combinations thereof.

Yet another embodiment of the present invention includes CLEC-6-specific antibodies or fragment thereof bound to one half of a Cohesin/Dockerin pair. The CLEC-6-specific antibody or fragment thereof may be bound to one half of a Cohesin/Dockerin pair and the complementary half may be bound to an antigen. The antigen may be a molecule, a peptide, a protein, a nucleic acid, a carbohydrate, a lipid, a cell, a virus or portion thereof, a bacteria or portion thereof, a fungi or portion thereof, a parasite or portion thereof. In another embodiment, the CLEC-6-specific antibody or fragment thereof is bound to one half of a Cohesin/Dockerin pair and the other half of the pair is bound to one or more cytokines selected from interleukins, transforming growth factors (TGFs), fibroblast growth factors (FGFs), platelet derived growth factors (PDGFs), epidermal growth factors (EGFs), connective tissue activated peptides (CTAPs), osteogenic factors, and biologically active analogs, fragments, and derivatives of such growth factors, B/T-cell differentiation factors, B/T-cell growth factors, mitogenic cytokines, chemotactic cytokines and chemokines, colony stimulating factors, angiogenesis factors, IFN-α, IFN-β, IFN-γ, IL1, IL2, IL3, IL4, IL5, IL6, IL7, IL8, IL9, IL10, IL 11, IL12, IL13, IL14, IL15, IL16, IL17, IL18, etc., leptin, myostatin, macrophage stimulating protein, platelet-derived growth factor, TNF-α, TNF-β, NGF, CD40L, CD137L/4-1 BBL, human lymphotoxin-β, G-CSF, M-CSF, GM-CSF, PDGF, IL-1α, IL1-β, IP-10, PF4, GRO, 9E3, erythropoietin, endostatin, angiostatin, VEGF, transforming growth factor (TGF) supergene family include the beta transforming growth factors (for example TGF-β1, TGF-β2, TGF-β3); bone morphogenetic proteins (for example, BMP-1, BMP-2, BMP-3, BMP-4, BMP-5, BMP-6, BMP-7, BMP-8, BMP-9); heparin-binding growth factors (fibroblast growth factor (FGF), epidermal growth factor (EGF), platelet-derived growth factor (PDGF), insulin-like growth factor (IGF)); Inhibins (for example, Inhibin A, Inhibin B); growth differentiating factors (for example, GDF-1); and Activins (for example, Activin A, Activin B, Activin AB).

The present invention also includes a method for separating myeloid dendritic cells from plasmacytoid dendritic cells by using CLEC-6 expression to isolate myeloid dendritic cells, B cells or monocytes that express CLEC-6 from plasmacytoid dendritic cells which do not express CLEC-6.

The invention includes a hybridoma that expressed a CLEC-6-specific antibody or fragment thereof, wherein the CLEC-6-specific antibody or fragment thereof activates an antigen presenting cell to express new surface markers, secrete one or more cytokines or both, for example, clone 12H7, 12E3, 9D5, 20H8 and combinations thereof. The antibodies produced by anti CELC-6 hybridomas may be used in a method for enhancing B cell immune responses by triggering a CLEC-6 receptor on a B cell to increase antibody production, secrete cytokines, increase B cell activation surface marker expression and combinations thereof. The B cells secrete IL-8, MIP- 1 a and combinations thereof and/or increases production of IgM, IgG and IgA.

The present invention also includes a method for enhancing T cell activation by triggering a CLEC-6 receptor on a dendritic cell with a CLEC-6 specific antibody or fragment and contacting a T cell to the CLEC-6 activated dendritic cell, wherein T cell activation is enhanced. The T cell may be a nayve CD8+T cell and the dendritic cells may be contacted with GM-CSF and IL-4, interferon alpha, antigen and combinations thereof. It has been found that the T-cells activated by the CLEC-6 activated DCs increases T cell secretion of IL-10, IL-15, and surface expression of 4-1BBL and combinations thereof. The T cells may also proliferate upon exposure to dendritic cells activated with anti-CLEC-6 antibodies or fragments thereof.

The present invention also includes an anti-CLEC-6 immunoglobulin or portion thereof that is secreted from mammalian cells and an antigen bound to the immunoglobulin. The anti- CELC-6 antigen specific domain may be a full length antibody, an antibody variable region domain, an Fab fragment, a Fab' fragment, an F(ab)2 fragment, and Fv fragment, and Fabc fragment and/or a Fab fragment with portions of the Fc domain. The anti-CELC-6 antibody may also be used to make a vaccine that includes a dendritic cell activated with a CLEC-6-specific antibody or fragment thereof.

The present invention also includes use of agents that engage the CLEC-6 receptor on immune cells, alone or with co-activating agents, the combination activating antigen-presenting cells for therapeutic applications; use of a CLEC-6 binding agent linked to one or more antigens, with or without activating agents, on immune cells to make a vaccine; use of anti-CLEC-6 agents as co-activating agents of immune cells for the enhancement of immune responses directed through a cell surface receptor other than CLEC-6 expressed on immune cells; use of anti-CLEC-6 antibody V-region sequences capable of binding to and activating immune cells through the CLEC-6 receptor and/or use of DC-CLEC-6 binding agents linked to one or more toxic agents for therapeutic purposes in the context of diseases known or suspected to result from inappropriate activation of immune cells via CLEC-6 or in the context of pathogenic cells or tissues that express CLEC-6.

Yet another embodiment includes a modular rAb carrier that includes a CLEC-6-specific antibody binding domain linked to one or more antigen carrier domains that comprise one half of a cohesin-dockerin binding pair. The antigen-specific binding domain may includes at least a portion of an antibody and/or at least a portion of an antibody in a fusion protein with the one half of the cohesin-dockerin binding pair. In one embodiment, the rAb may also include a complementary half of the cohesin-dockerin binding pair bound to an antigen that forms a complex with the modular rAb carrier, or a complementary half of the cohesin-dockerin binding pair that is a fusion protein with an antigen. The antigen specific domain of the rAb may be a full length antibody, an antibody variable region domain, an Fab fragment, a Fab' fragment, an F(ab)2 fragment, and Fv fragment, and Fabc fragment and/or a Fab fragment with portions of the Fc domain.

### BRIEF DESCRIPTION OF THE DRAWINGS

For a more complete understanding of the features and advantages of the present invention, reference is now made to the detailed description of the invention along with the accompanying figures and in which:

FIGS. 1A and 1B show both in vivo and in vitro-cultured DCs express CLEC-6. FIG. 1A shows PBMCs from normal donors were stained with anti-CD11c, CD 14, CD19, and CD3 with anti-CLEC-6 mAbs. Cells stained with individual antibodies were gated to measure the expression levels of CLEC-6. FIG. 1B shows monocytes from normal donors were cultured in the presence of GM-CSF with IL-4 (IL-4DCs) or IFNa (IFNDCs), and cells were stained with anti-CLEC-6 mAb or isotype control antibody. C. myeloid DCs (Lin-HLA-DR+CD11c+CD123-) were purified from blood by FACS sorter, and stained with anti-CLEC-6 mAbs. Open and closed histograms represent cells stained with, respectively, isotype control and anti-CLEC-6 mAb.

FIG. 2 shows that Anti-CLEC-6 mAbs activate DCs. IFNDCs (1×10⁵/200 ul/well) were cultured in the plates coated with different clones of mAbs for 18 h. Culture supernatants were analyzed to measure cytokines and chemokines by Luminex.

FIGS. 3A and 3B show that anti-CLEC-6 mAbs activate DCs. FIG. 3A shows IL-4DCs (1×10⁵/well/200 ul) stimulated with anti-CLEC-6 for 18 h, and then cells were stained with anti-CD86 and HLA-DR. FIG. 3B shows myeloid DCs purified from blood by FACS sorting. mDCs (1×10^{5/}well/200 ul) were stimulated with anti-CLEC-6 mAbs for 18 h, and cells were stained with anti-CD86, CD80, and HLA-DR.

FIG. 4 shows the gene expression profile for IL-4DCs stimulated with either anti-CLEC-6 or control mAbs for 12 h. Total RNA extracted with RNeasy columns (Qiagen), and analyzed with the 2100 Bioanalyser (Agilent). Biotin-labeled cRNA targets were prepared using the Illumina totalprep labeling kit (Ambion) and hybridized to Sentrix Human6 BeadChips (46K transcripts). These microarrays consist of 50 mer oligonucleotide probes attached to 3 um beads which are lodged into microwells etched at the surface of a silicon wafer. After staining with Streptavidin-Cy3, the array surface is imaged using a sub-micron resolution scanner manufactured by Illumina (Beadstation 500). A gene expression analysis software program, GeneSpring, Version 7.1 (Agilent), was used to perform data analysis.

FIGS. 5A and 5B show DCs activated with anti-CLEC-6 produce increased amounts of cytokines and chemokines. In vitro-cultured IL-4DCs and purified mDCs (1 10^{5 /200} ul), as described in FIG. 1 legend, were cultured in the plates coated with anti-CLEC-6 mAb (2 ug/well) for 18 h. Culture supernatants were analyzed to measure cytokine and chemokines by Luminex.

FIGS. 6A and 6B show that CLEC-6 and CD40 synergize to activate DCs. IL-4DCs (2 10⁵/200 ul/well) were cultured in the 96-well plates coated with anti-CLEC-6 in the presence or absence of soluble CD40L (20 ng/ml) for 18 h. Control mAbs were also tested. After 18 h, cells were stained with anti-CD83 and culture supernatants were analyzed to measure cytokines and chemokines by Luminex.

FIGS. 7A to 7C show that CLEC-6 expressed on DCs contributes to enhanced humoral immune responses. Six day GM/IL-4 DCs, 5 10³/well, were incubated in 96 well plates coated with anti-CLEC-6 or control mAbs for 16-18 h, and then 1 10⁵ autologous CD19+B cells stained with CFSE were co-cultured in the presence of 20 units/ml IL-2 and 50 nM CpG. FIG. 7A: on day six, cells were stained with fluorescently labeled antibodies. CD3+and 7-AAD+cells were gated out. CD38+and CFSE- cells were purified by FACS sorter and Giemsa staining was performed. FIG. 7B shows the culture supernatants on day thirteen were analyzed for total IgM, IgG, and IgA by sandwich ELISA. FIG. 7C shows that six day GM/IL-4 DCs cultured in mAb-coated plates for 48 h, and expression levels of APRIL were determined by intracellular staining of the cells. Dotted lines are cells stained with control antibody. Thin and thick lines represent cells incubated in the plates coated with anti-CLEC-6 or control mAb, respectively. Data are representative of two separate experiments using cells from three different normal donors each time.

FIGS. 8A and 8B show that CLEC-6 expressed on B cells contributes to B cell activation and immunoglobulin production. FIG. 8A shows CD19+B cells (2 10⁵/well/200 ul) were cultured in plates coated with the mAbs for 16-18 h, and then culture supernatants were analyzed for cytokines and chemokines by Luminex. FIG. 8B shows 1 10⁵ CD19+B cells were cultured in plates coated with the mAbs for thirteen days. Total Ig levels were measured by ELISA. Data are representative of two repeat experiments using cells from three different normal donors.

FIGS. 9A to 9E show that CLEC-6 expressed on DCs contributes to enhanced antigen specific T cell responses. FIG. 9A. 5 10³ of six day IFNDCs were cultured in the plates coated with anti-CLEC-6 or control mAbs for 16-18 h, and then purified allogeneic T cells were co-cultured. Cells were pulsed with ³[H]-thymidine, 1 uCi/well, for 18 h before harvesting. ³[H]-thymidine uptake was measured by a beta-counter. FIG. 9B. IL-4DCs (5 10³/well) were incubated in plates coated with the mAbs in the presence of 100 nM Flu M 1 peptide (HLA-A2 epitope) (upper two panels) or recombinant Flu M 1 protein (lower two panels) for 16 h. 2 10 6 purified autologous CD8 T cells were co-cultured for 7 days. On day two, 20 units/ml IL-2 and 10 units/ml of IL-7 were added to the culture. Cells were stained with anti-CD8 and Flu M 1 l-tetramer. FIG. 9C. IL-4DCs (5 10³/well) were incubated in plates coated with the mAbs in the presence of 20 uM Mart- 1 peptide (HLA-A2 epitope)(upper two panels) or recombinant Mart- 1 protein (lower two panels) for 16 h. 2 10⁶ purified autologous CD8 T cells were co-cultured for 10 days. On day two, 20 units/ml IL-2 and 10 units/ml of IL-7 were added to the culture. Cells were stained with anti-CD8 and Mart-1-tetramer. FIG. 9D. IL-4DCs were loaded with 10 nM of anti-CLEC-6-Mart-1 complex or control Ig-Mart-1 complex for 2 h. 2 10⁶ purified autologous CD8 T cells were co-cultured for 10 days. Cells were stained with anti-CD8 and Flu M 1 -specific tetramer. Cells in the lower two panels were stimulated with 20 ng/ml LPS from E. coli. FIG. 9E. Purified mDCs loaded with 10 nM of anti-CLEC-6-Flu HAI or control Ig-Flu HA 1 complexes for 2 h. 2 10⁶ purified autologous CD4 T cells labeled with CFSE were co-cultured for 7 days. Cells were stained with anti-CD4, and cell proliferation was measured by analyzing CFSE dilution. Cells in lower two panels were stimulated with 20 ng/ml LPS from E. coli.

FIG. 10 shows PBMC from non-human primates (Cynomolgus) were stained with anti-CLEC-6 mAb and antibodies to cell surface markers and analyzed by FACS.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The Dectin- 1 gene cluster contains lectin-like oxidized low-density lipoprotein receptor (LOX)-1, C-type lectin-like receptor (CLEC)-1 and 2, as well as MICL. CLEC-1 is expressed intracellularly when transfected into culture cells, and, therefore, requirement of some adaptor molecule was predicted for its surface expression (M. Colonna et al. Eur J Immunol 30 (2000), pp. 697-704). However, no cationic amino acid is present in its transmembrane portion. Instead, one tyrosine residue is present in its cytoplasmic portion, but the signaling effect through this tyrosine is unknown. CLEC-2 contains one DxYxxL (aspartic acid-any-tyrosine-any-any-leucine) motif in its cytoplasm and is expressed on the transfected cell surface. This motif is known to encourage efficient endocytosis and basolateral expression of ASGPR-1, and is highly homologous to the second tyrosine-based motif of dectin-1. In fact, Syk is recruited to the phosphotyrosine of CLEC-2, induced by its ligand, the snake venom rhodocytin (aggretin) (K. Suzuki-Inoue et al. Blood 107 (2006), pp. 542-549). This observation confirms the presence of a unique single YxxL sequence in C-type lectin receptors, which provides a docking site for Syk when tyrosine-phosphorylated. MICL (CLEC12A) has been identified as an ITIM-containing molecule homologous to dectin-1 and LOX-1 (A. S. Marshall et al. J Biol Chem 279 (2004), pp. 14792-14802). Its expression is primarily restricted to monocytes, granulocytes and immature DCs. Functionally, MICL recruits SHP-1 and 2 upon stimulation and an ITIM-dependent inhibitory effect has been observed using a chimeric receptor containing cytoplasmic MICL (A. S. Marshall et al. J Biol Chem 279 (2004), pp. 14792-14802). In a recent report, however, after ligation of MICL on immature DCs, an altered protein tyrosine phophorylation pattern as well as serine phosphorylation of p38 MAPK and ERK were observed, and, furthermore, CCR7 expression and cytokine production were noted without upregulation of maturation marker such as CD83, 86 and DC-LAMP (C. H. Chen et al. Blood 107 (2006), pp. 1459-1467). Indeed, such CCR7⁺ costimulation low semi-mature phenotype is considered to represent the steady-state migrating DCs (L. Oh 1 et al. Imunity 21 (2004), pp. 279-288). Though still uncharacterized, the genes coding for CLEC9A and CLEC12B are also located in the dectin-1 gene cluster (G. D. Brown, Nat Rev Immunol 6 (2006), pp. 33-43). CLEC12B contains ITIM in its cytoplasmic tail, while CLEC9A bears an ExYxxL (glutamic acid-any-tyrosine-any-any-leucine) sequence, which might act as an activation motif. The functions of these molecules remain to be investigated.

Arce et al., Eur. J. Immunol. (2004) identified and characterized the human CLEC-6 protein, related to mouse Mc1/Clecsf8.Human CLEC-6 codes for a type II membrane glycoprotein of 215 amino acids that belongs to the human calcium-dependent lectin family (C-type lectin). The CLEC-6 extracellular region shows a single carbohydrate recognition domain (CRD). Biochemical analysis of CLEC-6 on transiently transfected cells showed a glycoprotein of 30 kDa and cross-linking of the receptor leads to a rapid internalization suggesting that CLEC-6 is an endocytic receptor (Arce et al., 2004). Unlike CLEC-1,-2,-9A, -12A, and -12B, CLEC-6 does not contain a YxxL motif or other consensus signaling motifs. No study has been done to characterize the biological function of CLEC-6.

DCs can cross-present protein antigens (Rock K L Immunol Rev. 2005 Oct;207:166-83). In vivo, DCs take up antigens by the means of a number of receptors and present antigenic peptides in both class I and II. In this context, DC lectins, as pattern recognition receptors, contribute to the efficient uptake of antigens as well as cross-presentation of antigens.

As used herein, the term “modular rAb carrier” is used to describe a recombinant antibody system that has been engineered to provide the controlled modular addition of diverse antigens, activating proteins, or other antibodies to a single recombinant monoclonal antibody (mAb), in this case, an anti-CLEC-6 monoclonal antibody. The rAb may be a monoclonal antibody made using standard hybridoma techniques, recombinant antibody display, humanized monoclonal antibodies and the like. The modular rAb carrier can be used to, e.g., target (via one primary recombinant antibody against an internalizing receptor, e.g., a human dendritic cell receptor) multiple antigens and/or antigens and an activating cytokine to dendritic cells (DC). The modular rAb carrier may also be used to join two different recombinant mAbs end-to-end in a controlled and defined manner.

The antigen binding portion of the “modular rAb carrier” may be one or more variable domains, one or more variable and the first constant domain, an Fab fragment, a Fab' fragment, an F(ab)₂ fragment, and Fv fragment, and Fabc fragment and/or a Fab fragment with portions of the Fc domain to which the cognate modular binding portions are added to the amino acid sequence and/or bound. The antibody for use in the modular rAb carrier can be of any isotype or class, subclass or from any source (animal and/or recombinant).

In one non-limiting example, the modular rAb carrier is engineered to have one or more modular cohesin-dockerin protein domains for making specific and defined protein complexes in the context of engineered recombinant InAbs. The mAb is a portion of a fusion protein that includes one or more modular cohesin-dockerin protein domains carboxy from the antigen binding domains of the mAb. The cohesin-dockerin protein domains may even be attached post-translationally, e.g., by using chemical cross-linkers and/or disulfide bonding.

The term “antigen” as used herein refers to a molecule that can initiate a humoral and/or cellular immune response in a recipient of the antigen. Antigen may be used in two different contexts with the present invention: as a target for the antibody or other antigen recognition domain of the rAb or as the molecule that is carried to and/or into a cell or target by the rAb as part of a dockerin/cohesin-molecule complement to the modular rAb carrier. The antigen is usually an agent that causes a disease for which a vaccination would be advantageous treatment. When the antigen is presented on MHC, the peptide is often about 8 to about 25 amino acids. Antigens include any type of biologic molecule, including, for example, simple intermediary metabolites, sugars, lipids and hormones as well as macromolecules such as complex carbohydrates, phospholipids, nucleic acids and proteins. Common categories of antigens include, but are not limited to, viral antigens, bacterial antigens, fungal antigens, protozoal and other parasitic antigens, tumor antigens, antigens involved in autoimmune disease, allergy and graft rejection, and other miscellaneous antigens.

The modular rAb carrier is able to carry any number of active agents, e.g., antibiotics, anti-infective agents, antiviral agents, anti-tumoral agents, antipyretics, analgesics, anti-inflammatory agents, therapeutic agents for osteoporosis, enzymes, cytokines, anticoagulants, polysaccharides, collagen, cells, and combinations of two or more of the foregoing active agents. Examples of antibiotics for delivery using the present invention include, without limitation, tetracycline, aminoglycosides, penicillins, cephalosporins, sulfonamide drugs, chloramphenicol sodium succinate, erythromycin, vancomycin, lincomycin, clindamycin, nystatin, amphotericin B, amantidine, idoxuridine, p-amino salicyclic acid, isoniazid, rifampin, antinomycin D, mithramycin, daunomycin, adriamycin, bleomycin, vinblastine, vincristine, procarbazine, imidazole carboxamide, and the like.

Examples of anti-tumor agents for delivery using the present invention include, without limitation, doxorubicin, Daunorubicin, taxol, methotrexate, and the like. Examples of antipyretics and analgesics include aspirin, Motrin®, Ibuprofen®, naprosyn, acetaminophen, and the like.

Examples of anti-inflammatory agents for delivery using the present invention include, without limitation, include NSAIDS, aspirin, steroids, dexamethasone, hydrocortisone, prednisolone, Diclofenac Na, and the like.

Examples of therapeutic agents for treating osteoporosis and other factors acting on bone and skeleton include for delivery using the present invention include, without limitation, calcium, alendronate, bone GLa peptide, parathyroid hormone and its active fragments, histone H4-related bone formation and proliferation peptide and mutations, derivatives and analogs thereof.

Examples of enzymes and enzyme cofactors for delivery using the present invention include, without limitation, pancrease, L-asparaginase, hyaluronidase, chymotrypsin, trypsin, tPA, streptokinase, urokinase, pancreatin, collagenase, trypsinogen, chymotrypsinogen, plasminogen, streptokinase, adenyl cyclase, superoxide dismutase (SOD), and the like.

Examples of cytokines for delivery using the present invention include, without limitation, interleukins, transforming growth factors (TGFs), fibroblast growth factors (FGFs), platelet derived growth factors (PDGFs), epidermal growth factors (EGFs), connective tissue activated peptides (CTAPs), osteogenic factors, and biologically active analogs, fragments, and derivatives of such growth factors. Cytokines may be B/T-cell differentiation factors, B/T-cell growth factors, mitogenic cytokines, chemotactic cytokines, colony stimulating factors, angiogenesis factors, IFN- , IFN- , IFN- , IL 1 , IL2, IL3, IL4, IL5, IL6, IL7, IL8, IL9, IL10, IL11, IL12, IL13, IL14, IL15, IL16, IL17, IL18, etc., leptin, myostatin, macrophage stimulating protein, platelet-derived growth factor, TNF-α, TNF-β, NGF, CD40L, CD137L/4-1 BBL, human lymphotoxin-β, G-CSF, M-CSF, GM-CSF, PDGF, IL-1α, IL1-β, IP-10, PF4, GRO, 9E3, erythropoietin, endostatin, angiostatin, VEGF or any fragments or combinations thereof. Other cytokines include members of the transforming growth factor (TGF) supergene family include the beta transforming growth factors (for example TGF-β1, TGF-β2, TGF-β3); bone morphogenetic proteins (for example, BMP-1, BMP-2, BMP-3, BMP-4, BMP-5, BMP-6, BMP-7, BMP-8, BMP-9); heparin-binding growth factors (for example, fibroblast growth factor (FGF), epidermal growth factor (EGF), platelet-derived growth factor (PDGF), insulin-like growth factor (IGF)); Inhibins (for example, Inhibin A, Inhibin B); growth differentiating factors (for example, GDF-1); and Activins (for example, Activin A, Activin B, Activin AB).

Examples of growth factors for delivery using the present invention include, without limitation, growth factors that can be isolated from native or natural sources, such as from mammalian cells, or can be prepared synthetically, such as by recombinant DNA techniques or by various chemical processes. In addition, analogs, fragments, or derivatives of these factors can be used, provided that they exhibit at least some of the biological activity of the native molecule. For example, analogs can be prepared by expression of genes altered by site-specific mutagenesis or other genetic engineering techniques.

Examples of anticoagulants for delivery using the present invention include, without limitation, include warfarin, heparin, Hirudin, and the like. Examples of factors acting on the immune system include for delivery using the present invention include, without limitation, factors which control inflammation and malignant neoplasms and factors which attack infective microorganisms, such as chemotactic peptides and bradykinins.

Examples of viral antigens include, but are not limited to, e.g., retroviral antigens such as retroviral antigens from the human immunodeficiency virus (HIV) antigens such as gene products of the gag, pol, and env genes, the Nef protein, reverse transcriptase, and other HIV components; hepatitis viral antigens such as the S, M, and L proteins of hepatitis B virus, the pre-S antigen of hepatitis B virus, and other hepatitis, e.g., hepatitis A, B, and C, viral components such as hepatitis C viral RNA; influenza viral antigens such as hemagglutinin and neuraminidase and other influenza viral components; measles viral antigens such as the measles virus fusion protein and other measles virus components; rubella viral antigens such as proteins E 1 and E2 and other rubella virus components; rotaviral antigens such as VP7sc and other rotaviral components; cytomegaloviral antigens such as envelope glycoprotein B and other cytomegaloviral antigen components; respiratory syncytial viral antigens such as the RSV fusion protein, the M2 protein and other respiratory syncytial viral antigen components; herpes simplex viral antigens such as immediate early proteins, glycoprotein D, and other herpes simplex viral antigen components; varicella zoster viral antigens such as gpI, gpII, and other varicella zoster viral antigen components; Japanese encephalitis viral antigens such as proteins E, M-E, M-E-NS1, NS1, NS1-NS2A, 80% E, and other Japanese encephalitis viral antigen components; rabies viral antigens such as rabies glycoprotein, rabies nucleoprotein and other rabies viral antigen components. See Fundamental Virology, Second Edition, eds. Fields, B. N. and Knipe, D. M. (Raven Press, New York, 1991) for additional examples of viral antigens.

Antigenic targets that may be delivered using the rAb-DC/DC-antigen vaccines of the present invention include genes encoding antigens such as viral antigens, bacterial antigens, fungal antigens or parasitic antigens. Viruses include picornavirus, coronavirus, togavirus, flavirvirus, rhabdovirus, paramyxovirus, orthomyxovirus, bunyavirus, arenavirus, reovirus, retrovirus, papilomavirus, parvovirus, herpesvirus, poxvirus, hepadnavirus, and spongiform virus. Other viral targets include influenza, herpes simplex virus 1 and 2, measles, dengue, smallpox, polio or HIV. Pathogens include trypanosomes, tapeworms, roundworms, helminthes, malaria. Tumor markers, such as fetal antigen or prostate specific antigen, may be targeted in this manner. Other examples include: HIV env proteins and hepatitis B surface antigen. Administration of a vector according to the present invention for vaccination purposes would require that the vector-associated antigens be sufficiently non-immunogenic to enable long term expression of the transgene, for which a strong immune response would be desired. In some cases, vaccination of an individual may only be required infrequently, such as yearly or biennially, and provide long term immunologic protection against the infectious agent. Specific examples of organisms, allergens and nucleic and amino sequences for use in vectors and ultimately as antigens with the present invention may be found in U.S. Pat. No. 6,541,011, relevant portions incorporated herein by reference, in particular, the tables that match organisms and specific sequences that may be used with the present invention.

Bacterial antigens for use with the rAb vaccine disclosed herein include, but are not limited to, e.g., bacterial antigens such as pertussis toxin, filamentous hemagglutinin, pertactin, FIM2, FIM3, adenylate cyclase and other pertussis bacterial antigen components; diptheria bacterial antigens such as diptheria toxin or toxoid and other diptheria bacterial antigen components; tetanus bacterial antigens such as tetanus toxin or toxoid and other tetanus bacterial antigen components; streptococcal bacterial antigens such as M proteins and other streptococcal bacterial antigen components; gram-negative bacilli bacterial antigens such as lipopolysaccharides and other gram-negative bacterial antigen components, Mycobacterium tuberculosis bacterial antigens such as mycolic acid, heat shock protein 65 (HSP65), the 30 kDa major secreted protein, antigen 85A and other mycobacterial antigen components; Helicobacter pylori bacterial antigen components; pneumococcal bacterial antigens such as pneumolysin, pneumococcal capsular polysaccharides and other pneumococcal bacterial antigen components; haemophilus influenza bacterial antigens such as capsular polysaccharides and other haemophilus influenza bacterial antigen components; anthrax bacterial antigens such as anthrax protective antigen and other anthrax bacterial antigen components; rickettsiae bacterial antigens such as rompA and other rickettsiae bacterial antigen component. Also included with the bacterial antigens described herein are any other bacterial, mycobacterial, mycoplasmal, rickettsial, or chlamydial antigens. Partial or whole pathogens may also be: haemophilus influenza; Plasmodium falciparum; neisseria meningitidis; streptococcus pneumoniae; neisseria gonorrhoeae; salmonella serotype typhi; shigella; vibrio cholerae; Dengue Fever; Encephalitides; Japanese Encephalitis; lyme disease; Yersinia pestis; west nile virus; yellow fever; tularemia; hepatitis (viral; bacterial); RSV (respiratory syncytial virus); HPIV 1 and HPIV 3; adenovirus; small pox; allergies and cancers.

Fungal antigens for use with compositions and methods of the invention include, but are not limited to, e.g., candida fungal antigen components; histoplasma fungal antigens such as heat shock protein 60 (HSP60) and other histoplasma fungal antigen components; cryptococcal fungal antigens such as capsular polysaccharides and other cryptococcal fungal antigen components; coccidiodes fungal antigens such as spherule antigens and other coccidiodes fungal antigen components; and tinea fungal antigens such as trichophytin and other coccidiodes fungal antigen components.

Examples of protozoal and other parasitic antigens include, but are not limited to, e.g., plasmodium falciparum antigens such as merozoite surface antigens, sporozoite surface antigens, circumsporozoite antigens, gametocyte/gamete surface antigens, blood-stage antigen pf 155/RESA and other plasmodial antigen components; toxoplasma antigens such as SAG-1, p30 and other toxoplasmal antigen components; schistosomae antigens such as glutathione-S-transferase, paramyosin, and other schistosomal antigen components; leishmania major and other leishmaniae antigens such as gp63, lipophosphoglycan and its associated protein and other leishmanial antigen components; and trypanosoma cruzi antigens such as the 75-77 kDa antigen, the 56 kDa antigen and other trypanosomal antigen components.

Antigen that can be targeted using the rAb of the present invention will generally be selected based on a number of factors, including: likelihood of internalization, level of immune cell specificity, type of immune cell targeted, level of immune cell maturity and/or activation and the like. Examples of cell surface markers for dendritic cells include, but are not limited to, MHC class I, MHC Class II, B7-2, CD18, CD29, CD31, CD43, CD44, CD45, CD54, CD58, CD83, CD86, CMRF-44, CMRF-56, DCIR and/or DECTIN-1 and the like; while in some cases also having the absence of CD2, CD3, CD4, CD8, CD14, CD15, CD16, CD 19, CD20, CD56, and/or CD57. Examples of cell surface markers for antigen presenting cells include, but are not limited to, MHC class I, MHC Class II, CD40, CD45, B7-1, B7-2, IFN- receptor and IL-2 receptor, ICAM-1 and/or Fc receptor. Examples of cell surface markers for T cells include, but are not limited to, CD3, CD4, CD8, CD 14, CD20, CD11b, CD16, CD45 and HLA-DR.

Target antigens on cell surfaces for delivery includes those characteristic of tumor antigens typically will be derived from the cell surface, cytoplasm, nucleus, organelles and the like of cells of tumor tissue. Examples of tumor targets for the antibody portion of the present invention include, without limitation, hematological cancers such as leukemias and lymphomas, neurological tumors such as astrocytomas or glioblastomas, melanoma, breast cancer, lung cancer, head and neck cancer, gastrointestinal tumors such as gastric or colon cancer, liver cancer, pancreatic cancer, genitourinary tumors such cervix, uterus, ovarian cancer, vaginal cancer, testicular cancer, prostate cancer or penile cancer, bone tumors, vascular tumors, or cancers of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma and leukemia.

Examples of antigens that may be delivered alone or in combination to immune cells for antigen presentation using the present invention include tumor proteins, e.g., mutated oncogenes; viral proteins associated with tumors; and tumor mucins and glycolipids. The antigens may be viral proteins associated with tumors would be those from the classes of viruses noted above. Certain antigens may be characteristic of tumors (one subset being proteins not usually expressed by a tumor precursor cell), or may be a protein which is normally expressed in a tumor precursor cell, but having a mutation characteristic of a tumor. Other antigens include mutant variant(s) of the normal protein having an altered activity or subcellular distribution, e.g., mutations of genes giving rise to tumor antigens.

Specific non-limiting examples of tumor antigens include: CEA, prostate specific antigen (PSA), HER-2/neu, BAGE, GAGE, MAGE 1-4, 6 and 12, MUC (Mucin) (e.g., MUC-1, MUC-2, etc.), GM2 and GD2 gangliosides, ras, myc, tyrosinase, MART (melanoma antigen), Pmel 17(gp 100 ), GnT-V intron V sequence (N-acetylglucoaminyltransferase V intron V sequence), Prostate Ca psm, PRAME (melanoma antigen), -catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, and Ki-67. In addition, the immunogenic molecule can be an autoantigen involved in the initiation and/or propagation of an autoimmune disease, the pathology of which is largely due to the activity of antibodies specific for a molecule expressed by the relevant target organ, tissue, or cells, e.g., SLE or MG. In such diseases, it can be desirable to direct an ongoing antibody-mediated (i.e., a Th2-type) immune response to the relevant autoantigen towards a cellular (i.e., a Th 1 -type) immune response. Alternatively, it can be desirable to prevent onset of or decrease the level of a Th2 response to the autoantigen in a subject not having, but who is suspected of being susceptible to, the relevant autoimmune disease by prophylactically inducing a Thl response to the appropriate autoantigen. Autoantigens of interest include, without limitation: (a) with respect to SLE, the Smith protein, RNP ribonucleoprotein, and the SS-A and SS-B proteins; and (b) with respect to MG, the acetylcholine receptor. Examples of other miscellaneous antigens involved in one or more types of autoimmune response include, e.g., endogenous hormones such as luteinizing hormone, follicular stimulating hormone, testosterone, growth hormone, prolactin, and other hormones.

Antigens involved in autoimmune diseases, allergy, and graft rejection can be used in the compositions and methods of the invention. For example, an antigen involved in any one or more of the following autoimmune diseases or disorders can be used in the present invention: diabetes, diabetes mellitus, arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves ophthalmopathy, sarcoidosis, primary biliary cirrhosis, uveitis posterior, and interstitial lung fibrosis. Examples of antigens involved in autoimmune disease include glutamic acid decarboxylase 65 (GAD 65), native DNA, myelin basic protein, myelin proteolipid protein, acetylcholine receptor components, thyroglobulin, and the thyroid stimulating hormone (TSH) receptor. Examples of antigens involved in allergy include pollen antigens such as Japanese cedar pollen antigens, ragweed pollen antigens, rye grass pollen antigens, animal derived antigens such as dust mite antigens and feline antigens, histocompatiblity antigens, and penicillin and other therapeutic drugs. Examples of antigens involved in graft rejection include antigenic components of the graft to be transplanted into the graft recipient such as heart, lung, liver, pancreas, kidney, and neural graft components. The antigen may be an altered peptide ligand useful in treating an autoimmune disease.

As used herein, the term “epitope(s)” refer to a peptide or protein antigen that includes a primary, secondary or tertiary structure similar to an epitope located within any of a number of pathogen polypeptides encoded by the pathogen DNA or RNA. The level of similarity will generally be to such a degree that monoclonal or polyclonal antibodies directed against such polypeptides will also bind to, react with, or otherwise recognize, the peptide or protein antigen. Various immunoassay methods may be employed in conjunction with such antibodies, such as, for example, Western blotting, ELISA, RIA, and the like, all of which are known to those of skill in the art. The identification of pathogen epitopes, and/or their functional equivalents, suitable for use in vaccines is part of the present invention. Once isolated and identified, one may readily obtain functional equivalents. For example, one may employ the methods of Hopp, as taught in U.S. Pat. No. 4,554,101, incorporated herein by reference, which teaches the identification and preparation of epitopes from amino acid sequences on the basis of hydrophilicity. The methods described in several other papers, and software programs based thereon, can also be used to identify epitopic core sequences (see, for example, Jameson and Wolf, 1988; Wolf et al., 1988; U.S. Pat. No. 4,554,101). The amino acid sequence of these “epitopic core sequences” may then be readily incorporated into peptides, either through the application of peptide synthesis or recombinant technology.

The preparation of vaccine compositions that includes the nucleic acids that encode antigens of the invention as the active ingredient, may be prepared as injectables, either as liquid solutions or suspensions; solid forms suitable for solution in, or suspension in, liquid prior to infection can also be prepared. The preparation may be emulsified, encapsulated in liposomes. The active immunogenic ingredients are often mixed with carriers which are pharmaceutically acceptable and compatible with the active ingredient.

The term “pharmaceutically acceptable carrier” refers to a carrier that does not cause an allergic reaction or other untoward effect in subjects to whom it is administered. Suitable pharmaceutically acceptable carriers include, for example, one or more of water, saline, phosphate buffered saline, dextrose, glycerol, ethanol, or the like and combinations thereof. In addition, if desired, the vaccine can contain minor amounts of auxiliary substances such as wetting or emulsifying agents, pH buffering agents, and/or adjuvants which enhance the effectiveness of the vaccine. Examples of adjuvants that may be effective include but are not limited to: aluminum hydroxide, N-acetyl-muramyl-L-threonyl-D-isoglutamine (thr-MDP), N-acetyl-nor-muramyl-L-alanyl-D-isoglutamine, MTP-PE and RIBI, which contains three components extracted from bacteria, monophosporyl lipid A, trehalose dimycolate and cell wall skeleton (MPL+TDM+CWS) in a 2% squalene/Tween 80 emulsion. Other examples of adjuvants include DDA (dimethyldioctadecylammonium bromide), Freund's complete and incomplete adjuvants and QuilA. In addition, immune modulating substances such as lymphokines (e.g., IFN- , IL-2 and IL-12) or synthetic IFN- inducers such as poly I:C can be used in combination with adjuvants described herein.

Pharmaceutical products that may include a naked polynucleotide with a single or multiple copies of the specific nucleotide sequences that bind to specific DNA-binding sites of the apolipoproteins present on plasma lipoproteins as described in the current invention. The polynucleotide may encode a biologically active peptide, antisense RNA, or ribozyme and will be provided in a physiologically acceptable administrable form. Another pharmaceutical product that may spring from the current invention may include a highly purified plasma lipoprotein fraction, isolated according to the methodology, described herein from either the patients blood or other source, and a polynucleotide containing single or multiple copies of the specific nucleotide sequences that bind to specific DNA-binding sites of the apolipoproteins present on plasma lipoproteins, prebound to the purified lipoprotein fraction in a physiologically acceptable, administrable form.

Yet another pharmaceutical product may include a highly purified plasma lipoprotein fraction which contains recombinant apolipoprotein fragments containing single or multiple copies of specific DNA-binding motifs, prebound to a polynucleotide containing single or multiple copies of the specific nucleotide sequences, in a physiologically acceptable administrable form. Yet another pharmaceutical product may include a highly purified plasma lipoprotein fraction which contains recombinant apolipoprotein fragments containing single or multiple copies of specific DNA-binding motifs, prebound to a polynucleotide containing single or multiple copies of the specific nucleotide sequences, in a physiologically acceptable administrable form.

The dosage to be administered depends to a great extent on the body weight and physical condition of the subject being treated as well as the route of administration and frequency of treatment. A pharmaceutical composition that includes the naked polynucleotide prebound to a highly purified lipoprotein fraction may be administered in amounts ranging from 1 g to 1 mg polynucleotide and 1 g to 100 mg protein.

Administration of an rAb and rAb complexes a patient will follow general protocols for the administration of chemotherapeutics, taking into account the toxicity, if any, of the vector. It is anticipated that the treatment cycles would be repeated as necessary. It also is contemplated that various standard therapies, as well as surgical intervention, may be applied in combination with the described gene therapy.

Where clinical application of a gene therapy is contemplated, it will be necessary to prepare the complex as a pharmaceutical composition appropriate for the intended application. Generally this will entail preparing a pharmaceutical composition that is essentially free of pyrogens, as well as any other impurities that could be harmful to humans or animals. One also will generally desire to employ appropriate salts and buffers to render the complex stable and allow for complex uptake by target cells.

Aqueous compositions of the present invention may include an effective amount of the compound, dissolved or dispersed in a pharmaceutically acceptable carrier or aqueous medium. Such compositions can also be referred to as inocula. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions. The compositions of the present invention may include classic pharmaceutical preparations. Dispersions also can be prepared in glycerol, liquid polyethylene glycols, and mixtures thereof and in oils. Under ordinary conditions of storage and use, these preparations contain a preservative to prevent the growth of microorganisms.

Disease States. Depending on the particular disease to be treated, administration of therapeutic compositions according to the present invention will be via any common route so long as the target tissue is available via that route in order to maximize the delivery of antigen to a site for maximum (or in some cases minimum) immune response. Administration will generally be by orthotopic, intradermal, subcutaneous, intramuscular, intraperitoneal or intravenous injection. Other areas for delivery include: oral, nasal, buccal, rectal, vaginal or topical. Topical administration would be particularly advantageous for treatment of skin cancers. Such compositions would normally be administered as pharmaceutically acceptable compositions that include physiologically acceptable carriers, buffers or other excipients.

Vaccine or treatment compositions of the invention may be administered parenterally, by injection, for example, either subcutaneously or intramuscularly. Additional formulations which are suitable for other modes of administration include suppositories, and in some cases, oral formulations or formulations suitable for distribution as aerosols. In the case of the oral formulations, the manipulation of T-cell subsets employing adjuvants, antigen packaging, or the addition of individual cytokines to various formulation that result in improved oral vaccines with optimized immune responses. For suppositories, traditional binders and carriers may include, for example, polyalkylene glycols or triglycerides; such suppositories may be formed from mixtures containing the active ingredient in the range of 0.5% to 10%, preferably 1%-2%. Oral formulations include such normally employed excipients as, for example, pharmaceutical grades of mannitol, lactose, starch magnesium stearate, sodium saccharine, cellulose, magnesium carbonate, and the like. These compositions take the form of solutions, suspensions, tablets, pills, capsules, sustained release formulations or powders and contain 10%-95% of active ingredient, preferably 25-70%.

The antigen encoding nucleic acids of the invention may be formulated into the vaccine or treatment compositions as neutral or salt forms. Pharmaceutically acceptable salts include the acid addition salts (formed with free amino groups of the peptide) and which are formed with inorganic acids such as, for example, hydrochloric or phosphoric acids, or with organic acids such as acetic, oxalic, tartaric, maleic, and the like. Salts formed with the free carboxyl groups can also be derived from inorganic bases such as, for example, sodium, potassium, ammonium, calcium, or ferric hydroides, and such organic bases as isopropylamine, trimethylamine, 2-ethylamino ethanol, histidine, procaine, and the like.

Vaccine or treatment compositions are administered in a manner compatible with the dosage formulation, and in such amount as will be prophylactically and/or therapeutically effective. The quantity to be administered depends on the subject to be treated, including, e.g., capacity of the subject's immune system to synthesize antibodies, and the degree of protection or treatment desired. Suitable dosage ranges are of the order of several hundred micrograms active ingredient per vaccination with a range from about 0.1 mg to 1000 mg, such as in the range from about 1 mg to 300 mg, and preferably in the range from about 10 mg to 50 mg. Suitable regiments for initial administration and booster shots are also variable but are typified by an initial administration followed by subsequent inoculations or other administrations. Precise amounts of active ingredient required to be administered depend on the judgment of the practitioner and may be peculiar to each subject. It will be apparent to those of skill in the art that the therapeutically effective amount of nucleic acid molecule or fusion polypeptides of this invention will depend, inter alia, upon the administration schedule, the unit dose of antigen administered, whether the nucleic acid molecule or fusion polypeptide is administered in combination with other therapeutic agents, the immune status and health of the recipient, and the therapeutic activity of the particular nucleic acid molecule or fusion polypeptide.

The compositions can be given in a single dose schedule or in a multiple dose schedule. A multiple dose schedule is one in which a primary course of vaccination may include, e.g., 1-10 separate doses, followed by other doses given at subsequent time intervals required to maintain and or reinforce the immune response, for example, at 1-4 months for a second dose, and if needed, a subsequent dose(s) after several months. Periodic boosters at intervals of 1-5 years, usually 3 years, are desirable to maintain the desired levels of protective immunity. The course of the immunization can be followed by in vitro proliferation assays of peripheral blood lymphocytes (PBLs) co-cultured with ESAT6 or ST-CF, and by measuring the levels of IFN- released from the primed lymphocytes. The assays may be performed using conventional labels, such as radionucleotides, enzymes, fluorescent labels and the like. These techniques are known to one skilled in the art and can be found in U.S. Pat. Nos. 3,791,932, 4,174,384 and 3,949,064, relevant portions incorporated by reference.

The modular rAb carrier and/or conjugated rAb carrier-(cohesion/dockerin and/or dockerin-cohesin)-antigen complex (rAb-DC/DC-antigen vaccine) may be provided in one or more “unit doses” depending on whether the nucleic acid vectors are used, the final purified proteins, or the final vaccine form is used. Unit dose is defined as containing a predetermined-quantity of the therapeutic composition calculated to produce the desired responses in association with its administration, i.e., the appropriate route and treatment regimen. The quantity to be administered, and the particular route and formulation, are within the skill of those in the clinical arts. The subject to be treated may also be evaluated, in particular, the state of the subject's immune system and the protection desired. A unit dose need not be administered as a single injection but may include continuous infusion over a set period of time. Unit dose of the present invention may conveniently may be described in terms of DNA/kg (or protein/Kg) body weight, with ranges between about 0.05, 0.10, 0.15, 0.20, 0.25, 0.5, 1, 10, 50, 100, 1,000 or more mg/DNA or protein/kg body weight are administered. Likewise the amount of rAb-DC/DC-antigen vaccine delivered can vary from about 0.2 to about 8.0 mg/kg body weight. Thus, in particular embodiments, 0.4 mg, 0.5 mg, 0.8 mg, 1.0 mg, 1.5 mg, 2.0 mg, 2.5 mg, 3.0 mg, 4.0 mg, 5.0 mg, 5.5 mg, 6.0 mg, 6.5 mg, 7.0 mg and 7.5 mg of the vaccine may be delivered to an individual in vivo. The dosage of rAb-DC/DC-antigen vaccine to be administered depends to a great extent on the weight and physical condition of the subject being treated as well as the route of administration and the frequency of treatment. A pharmaceutical composition that includes a naked polynucleotide prebound to a liposomal or viral delivery vector may be administered in amounts ranging from 1 g to 1 mg polynucleotide to 1 g to 100 mg protein. Thus, particular compositions may include between about 1 g, 5 g, 10 g, 20 g, 30 g, 40 g, 50 g, 60 g, 70 g, 80 g, 100 g, 150 g, 200 g, 250 g, 500 g, 600 g, 700 g, 800 g, 900 g or 1,000 g polynucleotide or protein that is bound independently to 1 g, 5 g, 10 g, 20 g, 3.0 g, 40 g 50 g, 60 g, 70 g, 80 g, 100 g, 150 g, 200 g, 250 g, 500 g, 600 g, 700 g, 800 g, 900 g, 1 mg, 1.5 mg, 5 mg, 10 mg, 20 mg, 30 mg, 40 mg, 50 mg, 60 mg, 70 mg, 80 mg, 90 mg or 100 mg vector.

The present invention was tested in an in vitro cellular system that measures immune stimulation of human Flu-specific T cells by dendritic cells to which Flu antigen has been targeted. The results shown herein demonstrate the specific expansion of such antigen specific cells at doses of the antigen which are by themselves ineffective in this system.

The present invention may also be used to make a modular rAb carrier that is, e.g., a recombinant humanized mAb (directed to a specific human dendritic cell receptor) complexed with protective antigens from Ricin, Anthrax toxin, and Staphylococcus B enterotoxin. The potential market for this entity is vaccination of all military personnel and stored vaccine held in reserve to administer to large population centers in response to any biothreat related to these agents. The invention has broad application to the design of vaccines in general, both for human and animal use. Industries of interest include the pharmaceutical and biotechnology industries.

The present invention includes compositions and methods, including vaccines, that specifically target (deliver) antigens to antigen-presenting cells (APCs) for the purpose of eliciting potent and broad immune responses directed against the antigen. These compositions evoke protective or therapeutic immune responses against the agent (pathogen or cancer) from which the antigen was derived. In addition the invention creates agents that are directly, or in concert with other agents, therapeutic through their specific engagement of the CLEC-6 receptor that is expressed on antigen-presenting cells.

Materials and Methods

Antibodies and tetramers -Antibodies (Abs) for surface staining of DCs and B cells, including isotype control Abs, were purchased from BD Biosciences (CA). Abs for ELISA were purchased from Bethyl (TX). Anti-BLyS and anti-APRIL were from PeproTech (NJ). Tetramers, HLA-A*0201-GILGFVFTL (Flu M 1 ) and HLA-A*0201-ELAGIGILTV (Mart-1), were purchased from Beckman Coulter (CA).

Cells and cultures Monocytes ( 1 10 6/ml) from normal donors were cultured in Cellgenics (France) media containing GM-CSF (100 ng/ml) and IL-4 (50 ng/ml) (R&D, CA). For day three and day six, DCs, the same amounts of cytokines were supplemented into the media on day one and day three, respectively. B cells were purified with a negative isolation kit (BD). CD4 and CD8 T cells were purified with magnetic beads coated with anti-CD4 or CD8 (Milteniy, Calif.). PBMCs were isolated from Buffy coats using Percoll™ gradients (GE Healthcare UK Ltd, Buckinghamshire, UK) by density gradient centrifugation. For DC activation, 1 10⁵ DCs were cultured in the mAb-coated 96-well plate for 16-18 h. mAbs (1-2 ug/well) in carbonate buffer, pH 9.4, were incubated for at least 3 h at 37° C. Culture supernatants were harvested and cytokines / chemokines were measured by Luminex (Biorad, Calif.). For gene analysis, DCs were cultured in the plates coated with mAbs for 8 h. In some experiments, soluble 50 ng/ml of CD40L (R&D, CA) or 50 nM CpG (InVivogen, Calif.) was added into the cultures. In the DCs and B cell co-cultures, 5 10³ DCs resuspended in RPMI 1640 with 10% FCS and antibiotics (Biosource, CA) were first cultured in the plates coated with mAbs for at least 6 h, and then 1×10⁵ purified autologous B cells labeled with CFSE (Molecular Probes, Oreg.) were added. In some experiments, DCs were pulsed with 5 moi (multiplicity of infection) of heat-inactivated influenza virus (A/PR/8 H 1 N 1 ) for 2 h, and then mixed with B cells. For the DCs and T cell co-cultures, 5x10³ DCs were cultured with Ix10⁵ purified autologous CD8 T cells or mixed allogeneic T cells. Allogeneic T cells were pulsed with 1 uCi/well ³[H]-thymidine for the final 18 h of incubation, and then cpm were measured by a beta-counter (Wallac, Minn.). 5 10⁵ PBMCs /well were cultured in the plates coated with mAbs. The frequency of Mart- 1 and Flu M 1 specific CD8 T cells was measured by staining cells with anti-CD8 and tetramers on day ten and day seven of the cultures, respectively. 10 uM of Mart- 1 peptide (ELAGIGILTV)and 20 nM of recombinant protein containing Mart- 1 peptides (see below) were added to the DC and CD8 T cell cultures. 20 nM purified recombinant Flu M 1 protein (see below) was add to the PBMC cultures.

Monoclonal antibodies Mouse mAbs were generated by conventional technology. Briefly, six-week-old BALB/c mice were immunized i.p. with 20 g of receptor ectodomain.hIgGFc fusion protein with Ribi adjuvant, then boosts with 20 μg antigen ten days and fifteen days later. After three months, the mice were boosted again three days prior to taking the spleens. Alternately, mice were injected in the footpad with 1-10 g antigen in Ribi adjuvant every three to four days over a thirty to forty day period. Three to four days after a final boost, draining lymph nodes were harvested. B cells from spleen or lymph node cells were fused with SP2/O-Ag 14 cells. Hybridoma supernatants were screened to analyze Abs to the receptor ectodomain fusion protein compared to the fusion partner alone, or the receptor ectodomain fused to alkaline phosphatase (15). Positive wells were then screened in FACS using 293F cells transiently transfected with expression plasmids encoding full-length receptor cDNAs. Selected hybridomas were single cell cloned and expanded in CELLine flasks (Integra, Calif.). Hybridoma supernatants were mixed with an equal volume of 1.5 M glycine, 3 M NaCl, 1 PBS, pH 7.8 and tumbled with MabSelect resin. The resin was washed with binding buffer and eluted with 0.1 M glycine, pH 2.7. Following neutralization with 2 M Tris, mAbs were dialyzed versus PBS.

ELISA - Sandwich ELISA was performed to measure total IgM, IgG, and IgA as well as flu-specific immunoglobulins (Igs). Standard human serum (Bethyl) containing known amounts of Igs and human AB serum were used as standard for total Igs and flu-specific Igs, respectively. Flu specific Ab titers, units, in samples were defined as dilution factor of AB serum that shows an identical optical density. The amounts of BAFF and BLyS were measured by ELISA kits (Bender MedSystem, CA).

RNA purification and gene analysis - Total RNA extracted with RNeasy columns (Qiagen), and analyzed with the 2100 Bioanalyser (Agilent). Biotin-labeled cRNA targets were prepared using the Illumina totalprep labeling kit (Ambion) and hybridized to Sentrix Human6 BeadChips (46K transcripts). These microarrays consist of 50 mer oligonucleotide probes attached to 3um beads which are lodged into microwells etched at the surface of a silicon wafer. After staining with Streptavidin-Cy3, the array surface is imaged using a sub-micron resolution scanner manufactured by Illumina (Beadstation 500). A gene expression analysis software program, GeneSpring, Version 7.1 (Agilent), was used to perform data analysis.

Expression and purification of recombinant Flu M 1 and MART-1 proteins PCR was used to amplify the ORF of Influenza A/Puerto Rico/8/34/Mount Sinai (H 1 N 1 ) M 1 gene while incorporating an Nhe I site distal to the initiator codon and a Not I site distal to the stop codon. The digested fragment was cloned into pET28b(+) (Novagen), placing the M 1 ORF in-frame with a His6 tag, thus encoding His.Flu MI protein. A pET28b (+) derivative encoding an N-terminal 169 residue cohesin domain from C. thermocellum (unpublished) inserted between the Nco I and Nhe I sites expressed Coh.His. For expression of Cohesin-Flex-hMART-1-PeptideA-His, the sequence GACACCACCGAGGCCCGCCACCCCCACCCCCCCGTGACCACCCCCACCACCACCGA CCGGAAGGGCACCACCGCCGAGGAGCTGGCCGGCATCGGCATCCTGACCGTGATCC TGGGCGGCAAGCGGACCAACAACAGCACCCCCACCAAGGGCGAATTCTGCAGATA TCCATCACACTGGCGGCCG (SEQ ID NO.: 1)(encoding DTTEARHPHPPVTTPTTDRKGTTAEELAGIGILTVILGGKRTNNSTPTKGEFCRYPSHWR P (SEQ ID NO.:2) the shaded residues are the immunodominant HLA-A2-restricted peptide and the underlined residues surrounding the peptide are from MART-1) was inserted between the Nhe I and Xho I sites of the above vector. The proteins were expressed in E. coli strain BL21 (DE3) (Novagen) or T7 Express (NEB), grown in LB at 37° C. with selection for kanamycin resistance (40 μg/ml) and shaking at 200 rounds/min to mid log phase growth when 120 mg/L IPTG was added. After three hours, the cells were harvested by centrifugation and stored at 80° C. E. coli cells from each 1 L fermentation were resuspended in 30 ml ice-cold 50 mM Tris, 1 mM EDTA pH 8.0 (buffer B) with 0.1 ml of protease inhibitor Cocktail II (Calbiochem, CA). The cells were sonicated on ice 2×5 min at setting 18 (Fisher Sonic Dismembrator 60) with a 5 min rest period and then spun at 17,000 r.p.m. (Sorvall SA-600) for 20 min at 4° C. For His.Flu MI purification the 50 ml cell lysate supernatant fraction was passed through 5 ml Q Sepharose beads and 6.25 ml 160 mM Tris, 40 mM imidazole, 4 M NaCl pH 7.9 was added to the Q Sepharose flow through. This was loaded at 4 ml/min onto a 5 ml HiTrap chelating HP column charged with Ni++. The column-bound protein was washed with 20 mM NaPO₄, 300 mM NaCl pH 7.6 (buffer D) followed by another wash with 100 mM H₃COONa pH 4.0. Bound protein was eluted with 100 mM H₃COONa pH 4.0. The peak fractions were pooled and loaded at 4 ml/min onto a 5 ml HiTrap S column equilibrated with 100 mM H₃COONa pH 5.5, and washed with the equilibration buffer followed by elution with a gradient from 0-1 M NaCl in 50 mM NaPO₄ pH 5.5. Peak fractions eluting at about 500 mM NaCl were pooled. For Coh.Flu Ml.His purification, cells from 2 L of culture were lysed as above. After centrifugation, 2.5 ml of Triton X114 was added to the supernatant with incubation on ice for 5 min. After further incubation at 25° C. for 5 min, the supernatant was separated from the Triton XI 14 following centrifugation at 25° C. The extraction was repeated and the supernatant was passed through 5 ml of Q Sepharose beads and 6.25 ml 160 mM Tris, 40 mM imidazole, 4 M NaCl pH 7.9 was added to the Q Sepharose flow through. The protein was then purified by Ni⁺⁺ chelating chromatography as described above and eluted with 0-500 mM imidazole in buffer D.

Particular sequence corresponding to the L and H variable regions of an anti-CLEC-6 mAb The invention encompasses a particular amino acid sequence shown below corresponding to anti-CLEC-6 monoclonal antibody that is a desirable component (in the context of e.g., humanized recombinant antibodies) of therapeutic or protective products. The following are such sequences in the context of chimeric mouse V region (underlined) - human C region (bold) recombinant antibodies.

rAB-pIRES2[mAnti_hCLEC_6_9B9.2G12_Kv-V-hIgGK-C]

(SEQ ID NO:3)

DIQMTQTTSSLSASLGDRVTISCRASQDISNYLNWYQQKPDGTVKLLIYY
TSILQLGVPSRFSGSGSETDYSLTISNLEQEDIATYFCQQGDSLPFTFGS
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
rAB-pIRES2[mAnti_hCLEC_6_9B9.2G12_Hv-LV-hIgG4H-C]

(SEQ ID NO.:4)

QVTLKESGPGILQPSQTLSLTCSFSGFSLSTSGMSVGWIRQPSGKGLEWL
AHIWWNDDKYYNPVLKSRLTISKETSNNQVFLKIASVVSADTATYYCARF
YGNCLDYWGQGTTLTVSSAKTKGPSVFPLAPCSRSTSESTAALGCLVKDY
FPEPVTVSWNSGALTSGVHTFPAVLQSSGLYSLSSVVTVPSSSLGTKTYT
CNVDHKPSNTKVDKRVESKYGPPCPPCPAPEFEGGPSVFLFPPKPKDTLM
ISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLGKAS

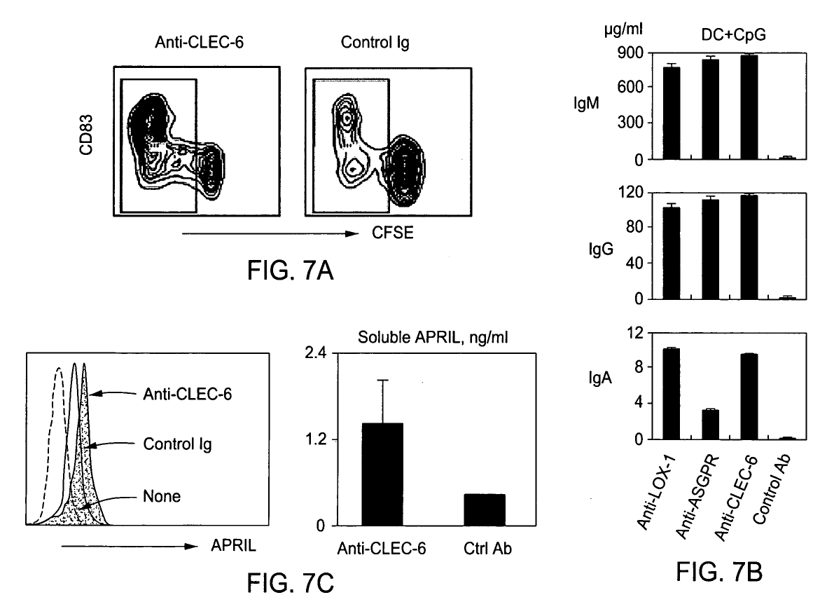

The present invention includes the use of the V-region sequences and related sequences modified by those well versed in the art to e.g., enhance affinity for CLEC-6 and/or integrated into human V-region framework sequences to be engineered into expression vectors to direct the expression of protein forms that can bind to CLEC-6 on antigen presenting cells. FIG. 7E shows engineered forms for use in, e.g., preclinical in vitro analysis). Furthermore, the other mAbs disclosed in the invention (or derived using similar methods and screens for the unique biology disclosed herein), can be via similar means (initially via PCR cloning and sequencing of mouse hybridoma V regions) be rendered into expression constructs encoding similar recombinant antibodies (rAbs). Such anti-CLEC-6 V regions can furthermore, by those well versed in the art, be ‘humanized (i.e., mouse—specific combining sequences grafted onto human V region framework sequences) so as to minimize potential immune reactivity of the therapeutic rAb.

Engineered recombinant anti-CLEC-6 recombinant antibody antigen fusion proteins (rAb.antigen) are efficacious prototype vaccines in vitro Expression vectors can be constructed with diverse protein coding sequence e.g., fused in-frame to the H chain coding sequence. For example, antigens such as Influenza HA5, Influenza M 1 , HIV gag, or immuno-dominant peptides from cancer antigens, or cytokines, can be expressed subsequently as rAb.antigen or rAb.cytokine fusion proteins, which in the context of this invention, can have utility derived from using the anti-CLEC-6 V-region sequence to bring the antigen or cytokine (or toxin) directly to the surface of the antigen presenting cell bearing CLEC-6. This permits internalization of e.g., antigen sometimes associated with activation of the receptor and ensuing initiation of therapeutic or protective action (e.g., via initiation of a potent immune response, or via killing of the targeted cell. A vaccine based on this concept could use a H chain vector encoding sequences such as those shown below cells. FIG. 7E above shows one example of the rAb for preclinical in vitro analysis:

rAB-pIRES2[mAnti_hCLEC_6_9B9.2G12(underlined)_
Hv-LV-hIgG4H-C(bold)-Flex-FluHA1-1-(italicized)
6xHis]

(SEQ ID NO.:5)

QVTLKESGPGILQPSQTLSLTCSFSGFSLSTSGMSVGWIRQPSGKGLEWL
AHIWWNDDKYYNPVLKSRLTISKETSNNQVFLKIASVVSADTATYYCARF
YGNCLDYWGQGTTLTVSSAKTKGPSVFPLAPCSRSTSESTAALGCLVKDY
FPEPVTVSWNSGALTSGVHTFPAVLQSSGLYSLSSVVTVPSSSLGTKTYT
CNVDHKPSNTKVDKRVESKYGPPCPPCPAPEFEGGPSVFLFPPKPKDTLM
ISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLGKASDTT
EPATPTTPVTTDTICIGYHANNSTDTVDTVLEKNVTVTHSVNLLEDSHNG
KLCRLKGIAPLQLGKCNIAGWLLGNPECDPLLPVRSWSYIVETPNSENGI
CYPGDFIDYEELREQLSSVSSFERFEIFPKESSWPNHNTNGVTAACSHEG
KSSFYRNLLWLTEKEGSYPKLKNSYVNKKGKEVLVLWGIHHPPNSKEQQN
LYQNENAYVSVVTSNYNRRFTPEIAERPKVRDQAGRMNYYWTLLKPGDTH
FEANGNLIAPMYAFALSRGFGSGHTSNASMHECNTKCQTPLGAINSSLPY
QNIHPVTIGECLKYVRSAKLRMVHHHHHH
rAB-pIRES2[mAnti_hCLEC_6_9B9.2G12_(underlined)
Hv-LV-hIgG4H-C-(bold)Flex-FluHA5-1-(italicized)
6xHis]

(SEQ ID NO.:6)

QVTLKESGPGILQPSQTLSLTCSFSGFSLSTSGMSVGWIRQPSGKGLEWL
AHIWWNDDKYYNPVLKSRLTISKETSNNQVFLKIASVVSADTATYYCARF
YGNCLDYWGQGTTLTVSSAKTKGPSVFPLAPCSRSTSESTAALGCLVKDY
FPEPVTVSWNSGALTSGVHTFPAVLQSSGLYSLSSVVTVPSSSLGTKTYT
CNVDHKPSNTKVDKRVESKYGPPCPPCPAPEFEGGPSVFLFPPKPKDTLM
ISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLGKASDTT
EPATPTTPVTTDQICIGYHANNSTEQVDTIMEKNVTVTHAQDILEKKHNG
KLCDLDGVKPLILRDCSVAGWLLGNPMCDEFINVPEWSYIVEKANPVNDL
CYPGDFNDYEELKHLLSRINHFEKIQHPKSSWSSHEASLGVSSACPYQGK
SSFFRNVVWLIKKNSTYPTIKRSYNNTNQEDLLVLWGIHHPNDAAEQTKL
YQNPTTYISVGTSTLNQRLVPRIATRSKVNGQSGRMEFFWTILKPNDAIN
FESNGNFIAPEYAYKIVKKGDSTIMKSELEYGNCNTKCQTPMGAINSSMP
FHNIHPLTIGECPKYVKSNRLVLAHHHHHH
rAB-pIRES2[mAnti_hCLEC_6_9B9.2G12_(underlined)
Hv-LV-hIgG4H-C(bold)-Dockerin(italicized)]

(SEQ ID NO.:7)

QVTLKESGPGILQPSQTLSLTCSFSGFSLSTSGMSVGWIRQPSGKGLEWL
AHIWWNDDKYYNPVLKSRLTISKETSNNQVFLKIASVVSADTATYYCARF
YGNCLDYWGQGTTLTVSSAKTKGPSVFPLAPCSRSTSESTAALGCLVKDY
FPEPVTVSWNSGALTSGVHTFPAVLQSSGLYSLSSVVTVPSSSLGTKTYT
CNVDHKPSNTKVDKRVESKYGPPCPPCPAPEFEGGPSVFLFPPKPKDTLM
ISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDG
SFFLYSRLTVDKSRWQEGNVFSCSVMHEALHNIIYTQKSLSLSLGKASNS
PQNEVLYGDVNDDGKVNSTDLTLLKRYVLKAVSTLPSSKAEKNADVNRDG
RVNSSDVTILSRYLIRVIEKLPI

Methods relating to the construction of prototype vaccines based on anti-CLEC-6 recombinant antibodies:

cDNA cloning and expression of chimeric mouse/human mAbs Total RNA was prepared from hybridoma cells (RNeasy kit, Qiagen) and used for cDNA synthesis and PCR (SMART RACE kit, BD Biosciences) using supplied 5′ primers and gene specific 3′ primers:

| | | |
|---|---|---|
| mIgGκ, | 5′ggatggtgggaagatggatacagttggtgcagcatc3′; | (SEQ ID NO.:8) |
| mIgGλ, | 5′ctaggaacagtcagcacgggacaaactcttctccacagtgtgaccttc3′; | (SEQ ID NO.:9) |
| mIgG1, | 5′gtcactggctcagggaaatagcccttgaccaggcatc3′; | (SEQ ID NO.:10) |
| mIgG2a, | 5′ccaggcatcctagagtcaccgaggagccagt3′; | (SEQ ID NO.:11) |
| and | | |
| mIgG2b, | 5′ggtgctggaggggacagtcactgagctgctcatagtgt3′. | (SEQ ID NO.:12) |

PCR products were cloned (pCR2.1 TA kit, Invitrogen) and characterized by DNA sequencing. Using the derived sequences for the mouse H and L chain V-region cDNAs, specific primers were used to PCR amplify the signal peptide and V-regions while incorporating flanking restriction sites for cloning into expression vectors encoding downstream human IgGK or IgG4H regions. The vector for expression of chimeric mVK-hlgK was built by amplifying residues 401-731 (gi 63101937) flanked by Xho I and Not I sites and inserting this into the Xho I Not I interval of pIRES2-DsRed2 (BD Biosciences). PCR was used to amplify the mAb Vk region from the initiator codon, appending a Nhe I or Spe I site then CACC, to the region encoding (e.g., residue 126 of gi 76779294), appending a Xho I site. The PCR fragment was then cloned into the Nhe I Not I interval of the above vector. The vector for chimeric mV -hIg using the mSLAM leader was built by inserting the sequence 5 ctagttgctggctaatggaccccaaaggctccctttcctggagaatacttctgtttctctccctggcttttgagttgtcgtacggattaattaag ggcccactcgag3′ (SEQ ID NO.: 13) into the Nhe I Xho I interval of the above vector. PCR was used to amplify the interval between the predicted mature N-terminal codon (defined using the SignalP 3.0 Server) (Bendtsen, Nielsen et al. 2004) and the end of the mV region (as defined above) while appending 5′tcgtacgga3′. The fragment digested with Bsi WI and Xho I was inserted into the corresponding sites of the above vector. The control hIg sequence corresponds to gi 49257887residues 26-85 and gi 21669402residues 67-709. The control hIgG4H vector corresponds to residues 12-1473 of gi 19684072with S229P and L236E substitutions, which stabilize a disulphide bond and abrogate residual FcR binding (Reddy, Kinney et al. 2000), inserted between the pIRES2-DsRed2 vector Bgl II and Not I sites while adding the sequence 5′gctagctgattaattaa3′ (SEQ ID NO.: 14) instead of the stop codon. PCR was used to amplify the mAb VH region from the initiator codon, appending CACC then a Bgl II site, to the region encoding residue 473 of gi|19684072|. The PCR fragment was then cloned into the Bgl II—Apa I interval of the above vector. The vector for chimeric mVH-hIgG4 sequence using the mSLAM leader was built by inserting the sequence 5′ ctagttgctggctaatggaccccaaaggctccctttcctggagaatacttctgtttctctccctggcttttgagttgtcgtacggattaattaag ggccc3′ (SEQ ID NO.: 15) into the Nhe I Apa I interval of the above vector. PCR was used to amplify the interval between the predicted mature N-terminal codon and the end of the mVH region while appending 5′tcgtacgga3′. The fragment digested with Bsi WI and Apa I was inserted into the corresponding sites of the above vector.

Various antigen coding sequences flanked by a proximal Nhe I site and a distal Not I site following the stop codon were inserted into the Nhe I Pac I Not I interval of the H chain vectors. Flu HA1- 1 was encoded by Influenza A virus (A/Puerto Rico/8/34(H 1 N 1 )) hemagglutinin gi 21693168 residues 82-1025 (with a C982T change) with proximal 5′gctagcgatacaacagaacctgcaacacctacaacacctgtaacaa3′ sequence (a Nhe I site followed by sequence encoding cipA cohesin-cohesin linker residues) and distal 5′caccatcaccatcaccattgagcggccgc3′ sequence (encoding His6, a stop codon, and a Not I site). Flu HA5-1 was encoded by gi 50296052 Influenza A virus (A/Viet Nam/1203/2004(H5N 1 )) hemagglutinin residues 49-990 bound by the same sequences as Flu HA 1 -1. Doc was encoded by gi 40671 celD residues 1923-2150 with proximal Nhe I and distal Not I sites. PSA was encoded by gi 34784812 prostate specific antigen residues 101-832 with proximal sequence 5′gctagcgatacaacagaacctgcaacacctacaacacctgtaacaacaccgacaacaacacttctagcgc3′(SEQ ID NO.:16)(Nhe I site and cipA spacer) and a distal Not I site. Flu M1-PEP was encoded by 5′ gctagccccattctgagccccctgaccaaaggcattctgggctttgtgtttaccctgaccgtgcccagcgaacgcaagggtatacttgga ttcgttttcacacttacttaagcggccgc3′ (SEQ ID NO.: 17). This and all other peptide-encoding sequences were created via mixtures of complimentary synthetic DNA fragments with ends compatible for cloning into Nhe I and Not I-restricted H chain vectors, or Nhe I Xho I-restricted Coh.His vector. Preferred human codons were always used, except where restriction sites needed to be incorporated or in CipA spacer sequences.

Production levels of rAb expression constructs were tested in 5 ml transient transfections using 2.5 g each of the L chain and H chain construct and the protocol described above. Supernatants were analyzed by anti-hIgG ELISA (AffiniPure Goat anti-human IgG (H+L), Jackson ImmunoResearch). In tests of this protocol, production of secreted rAb was independent of H chain and L chain vectors concentration over a 2-fold range of each DNA concentration (i.e., the system was DNA saturated).

The present invention includes the development, characterization and use of novel anti-human CLEC-6 reagents and their use to discover novel biology that is the basis of the invention and its envisioned applications. In summary, novel anti-CLEC-6 monoclonal antibodies (mAbs) were developed and used to uncover previously unknown biology associated with this cell surface receptor that is found on antigen-presenting cells. This novel biology is highly predictive of the application of anti-CLEC-6 agents that activate this receptor for diverse therapeutic and protective applications. Data presented below strongly support the initial predictions and demonstrate the pathway to reducing the discoveries revealed herein to clinical application.

Development of high affinity monoclonal antibodies against human CLEC-6Receptor ectodomain.hIgG (human IgGlFc) and AP (human placental alkaline phosphatase) fusion proteins were produced for immunization of mice and screening of mAbs, respectively. An expression construct for DCIR ectodomain.IgG was described previously (15) and used the mouse SLAM (mSLAM) signal peptide to direct secretion (16). A similar expression vector for hDCIR ectodomain.AP was generated using PCR to amplify AP resides 133-1581 (gb BC0096471) while adding a proximal in-frame Xho I site and a distal TGA stop codon and Not I site. This Xho I Not I fragment replaced the IgG coding sequence in the above DCIR ectodomain.IgG vector. CLEC-6 ectodomain constructs in the same Ig and AP vector series contained inserts encoding CLEC-6 (bp 317-838, gi 37577120 . CLEC-6 fusion proteins were produced using the FreeStyle™ 293 Expression System (Invitrogen) according to the manufacturer's protocol (1 mg total plasmid DNA with 1.3 ml 293 Fectin reagent/L of transfection). For rAb production, equal amounts of vector encoding the H and L chain were co-transfected. Transfected cells are cultured for 3 days, the culture supernatant was harvested and fresh media added with continued incubation for two days. The pooled supernatants were clarified by filtration. Receptor ectodomain.hIgG was purified by HiTrap protein A affinity chromatography with elution by 0.1 M glycine pH 2.7 and then dialyzed versus PBS. rAbs (recombinant antibodies described later)were purified similarly, by using HiTrap MabSelect™ columns. Mouse mAbs were generated by conventional cell fusion technology. Briefly, 6-week-old BALB/c mice were immunized intraperitonealy with 20 μg of receptor ectodomain.hIgGFc fusion protein with Ribi adjuvant, then boosts with 20 μg antigen 10 days and 15 days later. After 3 months, the mice were boosted again three days prior to taking the spleens. Alternately, mice were injected in the footpad with 1-10 g antigen in Ribi adjuvant every 3-4 days over a 30-40 day period. 3-4 days after a final boost, draining lymph nodes were harvested. B cells from spleen or lymph node cells were fused with SP2/O-Ag 14 cells (17) using conventional techniques. ELISA was used to screen hybridoma supernatants against the receptor ectodomain fusion protein compared to the fusion partner alone, or versus the receptor ectodomain fused to AP (15). Positive wells were then screened in FACS using 293F cells transiently transfected with expression plasmids encoding full-length receptor cDNAs. Selected hybridomas were single cell cloned, adapted to serum-free medium, and expanded in CELLine flasks (Intergra). Hybridoma supernatants were mixed with an equal volume of 1.5 M glycine, 3 M NaCl, 1 PBS, pH 7.8 and tumbled with MabSelect resin. The resin was washed with binding buffer and eluted with 0.1 M glycine, pH 2.7. Following neutralization with 2 M Tris, mAbs were dialyzed versus PBS.

Characterization of purified anti-CLEC-6 monoclonal antibodies by direct and indirect ELISA: The hybridoma clones were tested for relative affinities of several anti-CLEC-6 mAbs by ELISA (i.e., CLEC-6.Ig protein is immobilized on the microtiter plate surface and the antibodies are tested in a dose titration series for their ability to bind to CLEC-6.Ig (as detected by an anti-mouse IgG.HRP conjugate reagent). The panels are mAb reactivity to CLEC-6.Ig protein; (A and D), mAb reactivity to hIgGFc protein, and (B and E) mAb reactivity to CLEC-6.alkaline phosphatase fusion protein (C and F). In the latter case, the mAbs are plate bound (through an anti-mouse IgG reagent) and bind a constant amount of CLEC-6.AP in solution. The results show that the anti-CLEC-6 mAbs react specifically to CLEC-6 ectodomain with high affinity.

Characterization of purified anti-CLEC-6 monoclonal antibodies FACS versus 293F cells expressing full-length CLEC-6: Testing of the relative affinities of several anti-CLEC-6 mAbs was conducted by FACS (i.e., CLEC-6.mAbs at various concentrations are incubated with 293F cells expressing CLEC-6; after washing, cells were stained with anti-mouse IgG reagent derivatized with PE.; results are mean fluorescence intensity corrected for staining to 293F cells not expressing CLEC-6). The 4 mAbs shown all stain CLEC-6-bearing cells specifically, with a rank order of staining potency of 12H712E3>9D5>20H8.

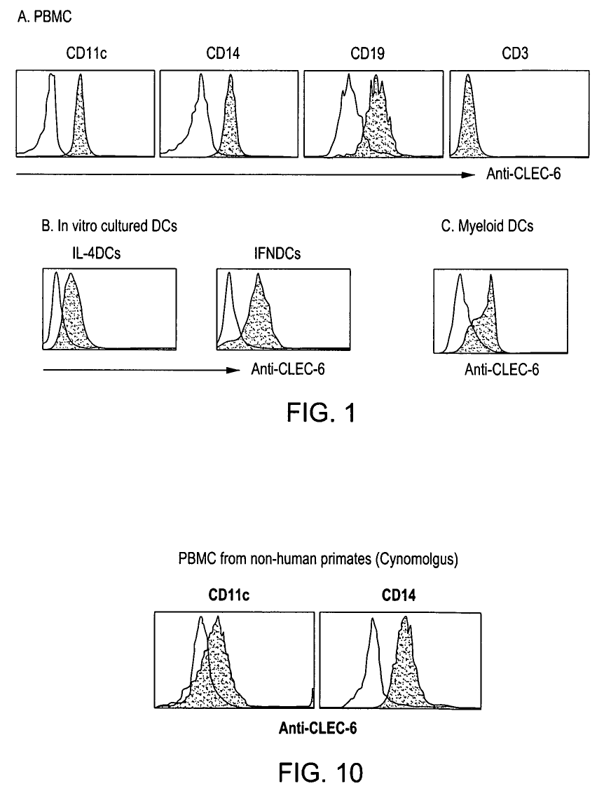

In vivo and in vitro-cultured DCs express CLEC-6The expression levels of CLEC-6 on PBMCs from normal donors was measure by FACS. As shown in FIG. 1 a , antigen presenting cells, including CD 11 c+DCs, CD14+monocytes, and CD19+B cells express CLEC-6. However, CD3+T cells do not express CLEC-6. CD56+NK cells did not express CLEC-6 (data not shown). Expression levels of CLEC-6 on in vitro-cultured DCs, as well as purified blood myeloid (mDCs) and plasmacytoid DCs (pDCs) were also determined. Data in FIG. 1 b show that both IL-4DCs and IFNDCs express significant levels of CLEC-6. The expression of CLEC-6 on in vitro cultured DCs is significant since it permits use of these cells in experiments directed to uncovering the function of CLEC-6. mDCs also express high levels of CLEC-6, but pDCs do not express CLEC-6 (data not shown). The latter observations are particularly important since they apply to cells isolated directly from blood and show that CLEC-6 is not present on all DC types thus suggesting that biology directed through CLEC-6 can address specific DC types, which are known to have different immune functions.

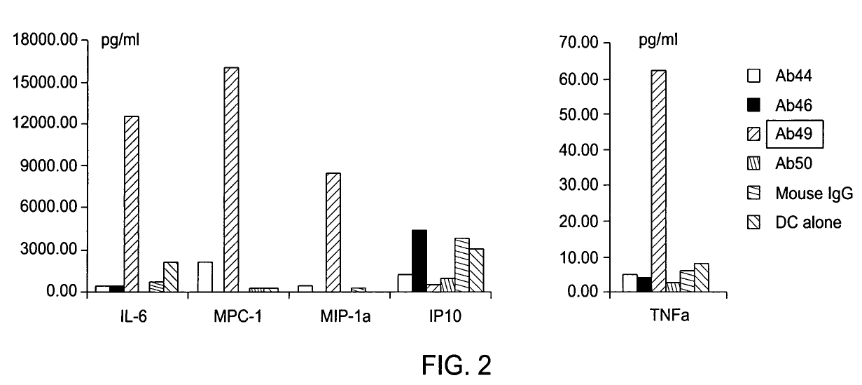

Selection of anti-CLEC-6 mAbs that can activate DCs-12 different hybridoma clones that produce mouse anti-human CLEC-6 mAbs were isolated and the mAbs they produce were tested for ability to activate DCs by measuring DC phenotypes and cytokines and chemokines secreted from DCs. Data in FIG. 2 show an example permitting identification of such mAbs that activate DCs. Of four anti-CLEC-6 mAbs, Ab49 could activate DCs and induce DCs to produce significant amounts of secreted IL-6, MIP- 1 a, MCP-1, IP-10, and TNFa. These anti-CLEC-6 mAbs also stimulate DCs to produce IL-12p40, IL- 1 a, and IL- 1 b (data not shown). Three other anti-CLEC-6 mAbs also activate DCs, and each mAb stimulates DCs to produce different levels of cytokines and chemokines.

These data demonstrate that only certain high affinity anti-CLEC-6 mAbs can activate human DC a previously unknown biology. This ability to elicit cytokine secretion by DC suggests such anti-CLEC-6 agents could influence immune responses in vivo.

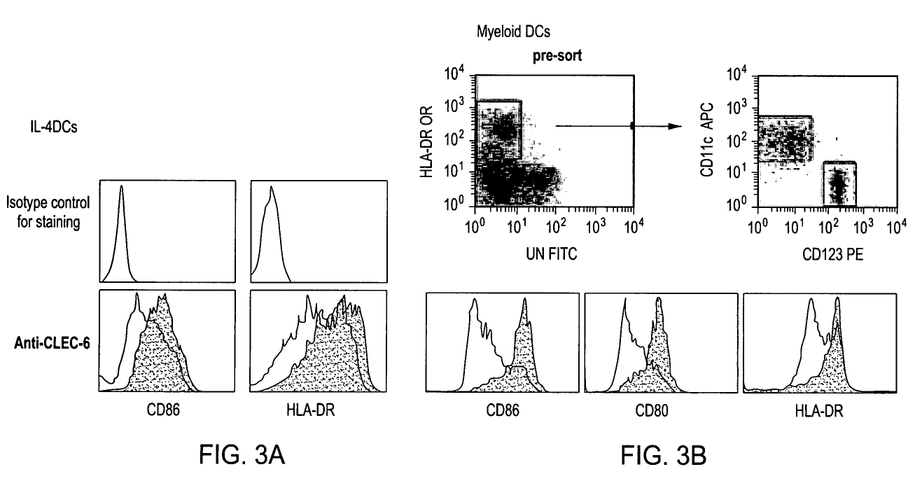

Signaling through CLEC-6 activates DC cell surface markers-DCs are the primary immune cells that determine the results of immune responses, either induction or tolerance, depending on their activation (18). Some of anti-CLEC-6 mAbs generated in this study could activate in vitro-cultured IFNDCs (FIG. 2), the role of CLEC-6 in the activation of different subsets of DCs (IL-4DCs and blood mDCs. IL-4DCs) was also tested. IL-4DCs were stimulated with anti-CLEC-6 mAb, and the data in FIG. 3a show that signals through CLEC-6 activate IL-4DCs, resulting in increased expression of cell surface markers CD86 and HLA-DR. Anti-CLEC-6 mAbs also activate in vivo DCs purified mDCs were stimulated with anti-CLEC-6 for 18 h, and then cells were stained with anti-CD86, CD80, and HLA-DR. As shown in FIG. 3 b , anti-CLEC-6 mAbs activate mDCs to express increased levels of CD86, CD80, and HLA-DR. The data in FIG. 3A and 3B demonstrate DC activation by specific anti-CLEC-6 mAbs to include up-regulation of cell surface molecules that are well known to be important in DC function.

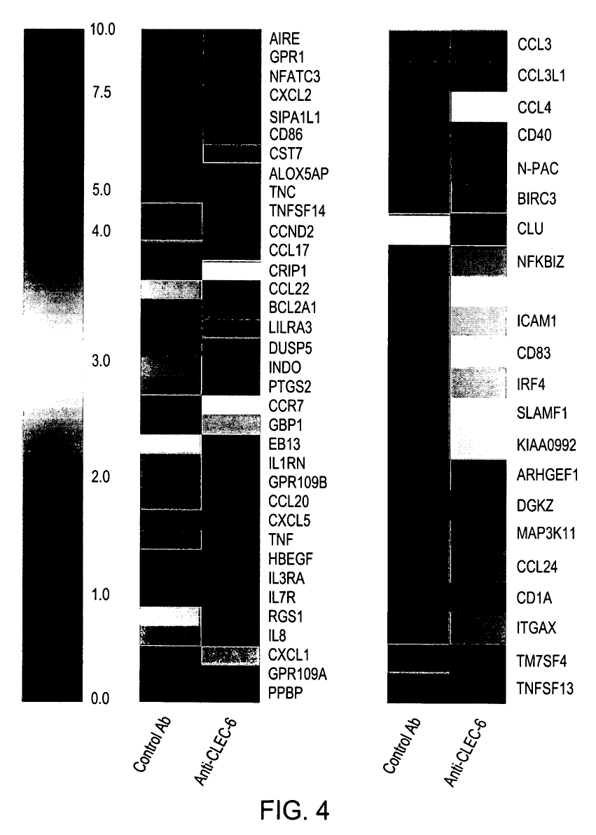

Signaling through CLEC-6 specific activates DC genes Consistently, DCs stimulated with anti-CLEC-6 mAbs express increased levels of multiple genes, including co-stimulatory molecules as well as chemokine and cytokine-related genes (FIG. 4). Compared to signals through other lectins, including DC-ASGPR and LOX-1 (data not shown), anti-CLEC-6 mAbs activate DCs in a unique fashion, suggesting that DCs activated through CLEC-6 should result in unique humoral and cellular immune responses.

Signaling through CLEC-6 activates genes in different DC subsets Both in vitro cultured IL-4DCs and mDCs produce significantly increased amounts of secreted IL-12p40, MCP-1, and IL-8 when they were stimulated with anti-CLEC-6 mAbs. Increased levels of other cytokines and chemokines, including TNFa, IL-6, MIP- 1 a, IL- 1 a, and IL- 1 b, were also observed in the culture supernatants of DCs stimulated with anti-CLEC-6 (not shown). Such cytokines are well known to be key mediators of immune responses and the discovery that specific anti-CLEC-6 agents elicit their production provides context to likely therapeutic application of such agents.

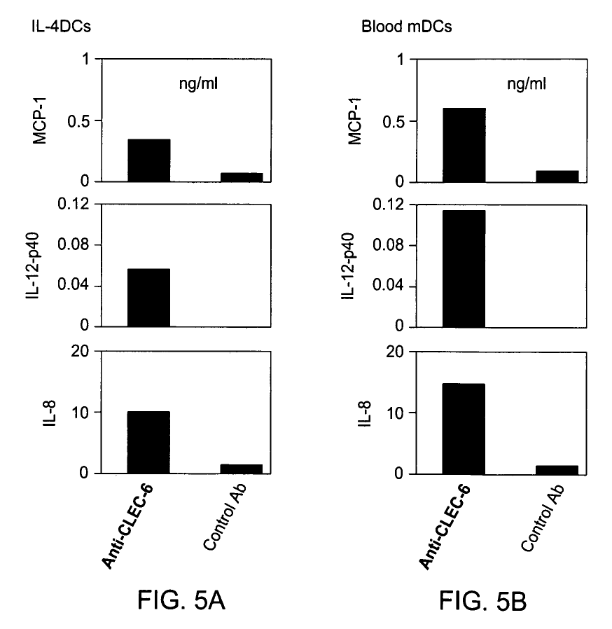

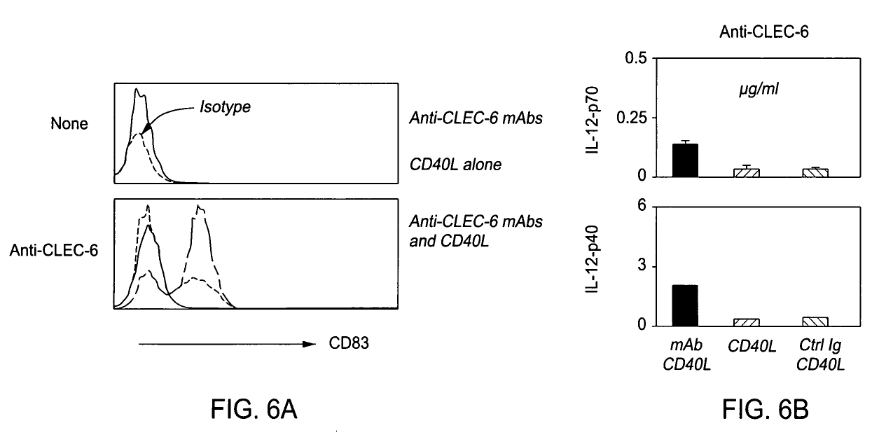

Signaling through CLEC-6 augments signaling through CD40- Signals through CLEC-6 synergize with the signal through CD40 for enhanced activation of DCs (FIG. 6). CLEC-6 engagement during CD40-CD40L interaction results in dramatically increased expression of cell surface CD83 (FIG. 6A) and production of secreted IL-12p70 and IL-12p40 (FIG. 6B). Other cytokines and chemokines, including TNFa, IL-6, MCP-1, MIP- 1 a, IL- 1 a, and IL- 1 b were also significantly increased (not shown). This is important because CLEC-6 can serve as a co-stimulatory molecule during in vivo DC activation. Taken together, data presented from FIG. 1 to FIG. 6 prove that signaling through CLEC-6 can activate DCs and that CLEC-6 serves as a potent co-stimulatory molecule for the activation of DCs.

DCs stimulated through CLEC-6 induce potent humoral immune responses-DCs play an important role in humoral immune responses by providing signals for both T-dependent and T-independent B cell responses (20-23) and by transferring antigens to B cells (24, 25). In addition to DCs, signaling through TLR9 as a third signal is necessary for efficient B cell responses (26, 27). Therefore, we tested the role of CLEC-6 in DCs-mediated humoral immune responses in the presence of TLR9 ligand, CpG. Six day GM/IL-4 DCs were stimulated with anti-CLEC-6 mAb, and then purified B cells were co-cultured. As shown in FIG. 7 a , DCs activated with anti-CLEC-6 mAb resulted in remarkably enhanced B cell proliferation (measured via CFSE dilution) and plasma cell differentiation (increase in the CD38⁺CD20⁻ population), compared to DCs stimulated with control mAb in the presence of CpG. CD38⁺CD20⁻ B cells have a typical morphology of plasma cells, but they do not express CD138 (data not shown). The majority of proliferating cells do not express CCR2, CCR4, CCR6, or CCR7 (data not shown).

The amounts of total immunoglobulins (Igs) produced were measured by ELISA (FIG. 7 b ). Anti-CLEC-6 was compared with mAbs to other lectins, LOX-1 and DC-ASGPR. Consistent with the data in FIG. 7a, B cells cultured with anti-CLEC-6-stimulated DCs to significantly increase production of total IgM, IgG, and IgA. DCs stimulated with anti-LOX-1 resulted in similar levels of IgM, IgG, and IgA productions from B cells. Unlike DCs stimulated with anti-CLEC-6 and anti-LOX-1 mAbs, DCs stimulated with anti-DC-ASGPR mAb resulted in significantly decreased amounts of IgG and IgA, suggesting that signals through CLEC-6 and LOX-1 induce B cell immunoglobulin class-switching. In addition to the total Igs, DCs activated by triggering LOX-1 are more potent than DCs stimulated with control mAb for the production of influenza-virus-specific IgM, IgG, and IgA (data not shown).

The mechanism by which DCs activated with anti-CLEC-6 result in the enhanced B cell responses involves a proliferation-inducing ligand (APRIL). DC-derived B lymphocyte stimulator protein (BLyS, BAFF) and APRIL are important molecules by which DCs can directly regulate human B cell proliferation and function (28-3 1). Data in FIG. 7c show that DCs stimulated through CLEC-6 expressed increased levels of intracellular APRIL as well as secreted APRIL, but not BLyS (not shown). Expression levels of BLyS and APRIL receptors on B cells in the mixed cultures were measured, but there was no significant change (not shown).

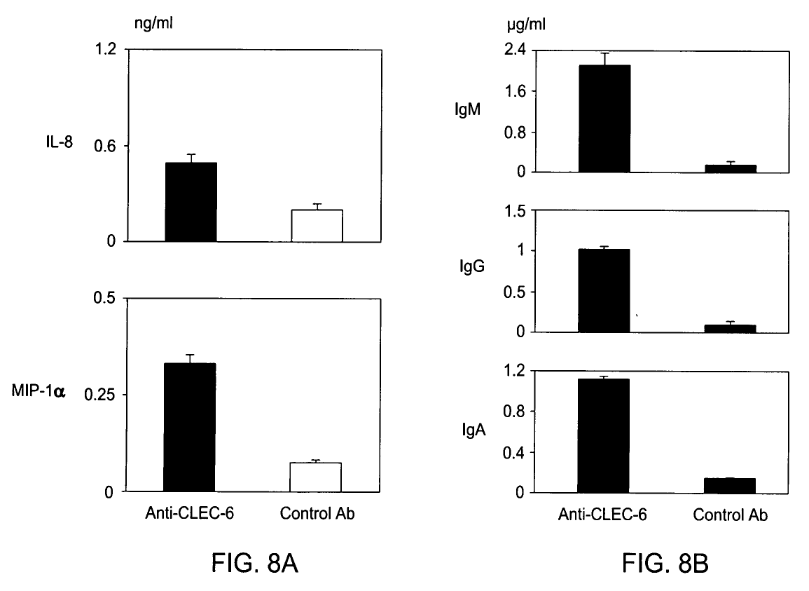

Anti-CLEC-6 mAbs have direct effects on human B cells CD19+B cells express CLEC-6 (FIG. 1) suggesting a role for CLEC-6 in B cell biology. Data in FIG. 8 a show that triggering CLEC-6 on B cells results in increased production of secreted IL-8 and MIP- 1 a, showing that CLEC-6 can also contribute to B cell activation. In addition to IL-8 and MIP- 1 a, slight increases in IL-6 and TNFa were also observed when B cells were stimulated with the anti-CLEC-6 mAb, compared to control mAb (not shown). B cells activated with anti-CLEC-6 mAb secreted increased amounts of total IgG, IgM, and IgA (FIG. 8 b ).

These observations demonstrate the direct action of CLEC-6 in the above studies of indirect effects (i.e., acting through DC) of anti-CLEC-6 agents on B cell biology. Taken together, these data reveal a high likelihood that such agents administered in vivo will stimulate antibody production e.g., as an adjuvant in vaccination, or (as is shown below) as a direct vehicle for targeting antigens to DC and other antigen presenting cells to elicit potent antigen-specific antibody responses.

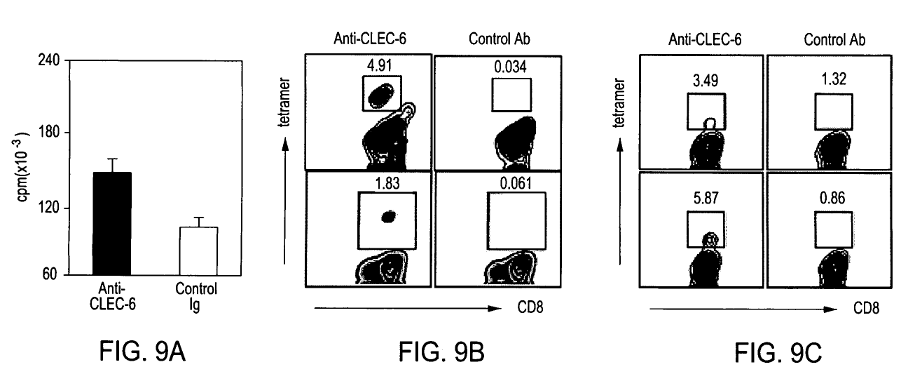

Role of CLEC-6 in T cell responses-DCs stimulated through CLEC-6 express enhanced levels of co-stimulatory molecules and produce increased amounts of cytokines and chemokines (FIG. 1, 2, and 3), suggesting that CLEC-6 contributes to cellular immune responses as well as humoral immune responses. This was tested by a mixed lymphocyte reaction (MLR). Proliferation of purified allogeneic T cells was significantly enhanced by DCs stimulated with mAb specific for CLEC-6 (FIG. 9 a ).

DCs activated through CLEC-6 also result in enhanced Flu M 1 specific CD8 T cell responses when DCs are pulsed with HLA-A2 epitope of Flu M 1 (upper two panels in FIG. 9B) as well as recombinant Flu M 1 protein (Lower two panels in FIG. 9B), suggesting that DCs activated with anti-CLEC-6 enhance cross-presentation of protein antigens. For therapeutic applications such as vaccine, it would be beneficial if signaling through CLEC-6 results in alterations of the capacity of DCs for naive CD8 T cell priming and cross-priming. Indeed, data in FIG. 9C show that DCs activated with anti-CLEC-6 mAb result in significantly enhanced Mart- 1 specific CD8 T cell priming (upper two panels in FIG. 9C) as well as cross-priming (lower two panels in FIG. 9C). Taken together, the data in FIG. 9A, B, and C indicates that CLEC-6 plays an important role in enhancing DC functions, resulting in the enhanced antigen specific CD8 T cell responses.

To validate the potential utility of CLEC-6 in a vaccine context, anti-CLEC-6 rAb-antigen complexes were compared with control rAb-antigen complexes for antigen-specific CD8 T cell responses. IFNDCs were loaded with 10 nM of the rAb-Mart-1 fusion proteins, and autologous CD8 T cells were co-cultured for 10 days. Cells were then stained with anti-CD8 and Mart- 1 tetramer. Data in FIG. 9 d show that anti-CLEC-6 rAb-antigen induced significantly enhanced Mart-I specific CD8 T cell responses compared to control (upper two panels in FIG. 9D). Data in the lower two panels in FIG. 9D was generated in the presence of 20 ng/ml LPS (from E. coli ). To further test the application of anti-CLEC-6 rAbs for vaccine application, mDCs were loaded with anti-CLEC-6-Flu HAlcomplexes or control rAb-Flu HA 1 complexes. Purified autologous CD4 T cells were co-cultured for 7 days, and then HAl-specific CD4 T cell proliferation appraised by measuring CFSE dilution. As shown in FIG. 9E (upper two panels), anti-CLEC-6 rAb- HAlinduced greater HAI-specific CD4 T cell proliferation than control rAb-HA1. Data in the lower two panels in FIG. 9E was generated in the presence of 20 ng/ml LPS (from E. coli ) which masks the CLEC-6-specific effect.

The data shown below serve as preclinical validation of using anti-CLEC-6-antigen complexes for vaccination purposes. Taken together they show that such prototype vaccines can direct antigen to target DC, and presumably together with associated activation through engaging CLEC-6, to take up, process, and present antigen to specific memory and nayve T cells and elicit their subsequent expansion. This property alone is sufficient to elicit antigen-specific cellular responses that are key components of cancer vaccines (to kill the cancer cells) or viral vaccines (to clear infected cells). Furthermore, the expansion of HA1-specific CD4 cells teaches that the anti-CLEC-6 prototype vaccine expands the type of T cell population that is key to eliciting antigen-specific humoral (antibody) responses. Data above show that the action of anti-CLEC-6 agents on Ig class switching further reinforces the high potential unique properties of such vaccines.

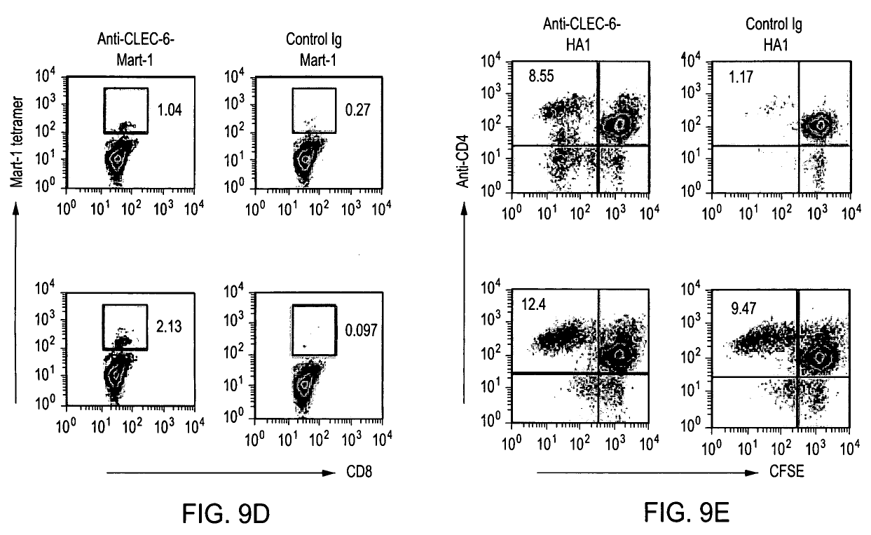

In vivo DCs in non-human primate express CLEC-6To test whether blood DCs in non-human primates (Cynomolgus) are reactive to the anti-human CLEC-6 mAbs, monkey PBMC were stained with anti-CLEC-6 mAbs and antibodies to other cellular markers, CD3, CD14, CD 11 c, CD27, CD56, and CD16. Data in FIG. 10 show that both CD14 and CD 11 c+cells were stained with anti-LOX-1 mAbs. However, CD3+, CD16+, CD27+, and CD56+cells did not express CLEC-6.

These data are important since validate monkey as a relevant model for pre-clinical studies of efficacy and safety of the diverse therapeutic anti-CLEC-6 agents that are envisioned in this invention.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims. REFERENCES

1. Figdor, C. G., Y. van Kooyk, and G. J. Adema. 2002. C-type lectin receptors on dendritic cells and Langerhans cells. Nat Rev Immunol 2:77-84.

2. Pyz, E., A. S. Marshall, S. Gordon, and G. D. Brown. 2006. C-type lectin-like receptors on myeloid cells. Ann Med 38:242-251.

3. Brown, G. D. 2006. Dectin-1: a signalling non-TLR pattern-recognition receptor. Nat Rev Immunol 6:33-43.

4. Geijtenbeek, T. B., D. J. Krooshoop, D. A. Bleijs, S. J. van Vliet, G. C. van Duijnhoven, V. Grabovsky, R. Alon, C. G. Figdor, and Y. van Kooyk. 2000. DC-SIGN-ICAM-2 interaction mediates dendritic cell trafficking. Nat Immunol 1 :353-357.

5. Geijtenbeek, T. B., R. Torensma, S. J. van Vliet, G. C. van Duijnhoven, G. J. Adema, Y. van Kooyk, and C. G. Figdor. 2000. Identification of DC-SIGN, a novel dendritic cell-specific ICAM-3 receptor that supports primary immune responses. Cell 100:575-585.

6. d'Ostiani, C. F., G. Del Sero, A. Bacci, C. Montagnoli, A. Spreca, A. Mencacci, P. Ricciardi-Castagnoli, and L. Romani. 2000. Dendritic cells discriminate between yeasts and hyphae of the fungus Candida albicans. Implications for initiation of T helper cell immunity in vitro and in vivo. J Exp Med 191:1661-1674.

7. Fradin, C., D. Poulain, and T. Jouault. 2000. beta-1,2-linked oligomannosides from Candida albicans bind to a 32-kilodalton macrophage membrane protein homologous to the mammalian lectin galectin-3. Infect Immun 68:4391-4398.

8. Cambi, A., K. Gijzen, J. M. de Vries, R. Torensma, B. Joosten, G. J. Adema, M. G. Netea, B. J. Kullberg, L. Romani, and C. G. Figdor. 2003. The C-type lectin DC-SIGN (CD209) is an antigen-uptake receptor for Candida albicans on dendritic cells. Eur J Immunol 33:532-538.

9. Netea, M. G., J. W. Meer, I. Verschueren, and B. J. Kullberg. 2002. CD40/CD40 ligand interactions in the host defense against disseminated Candida albicans infection: the role of macrophage-derived nitric oxide. Eur J Immunol 32:1455-1463.

10. Lee, S. J., S. Evers, D. Roeder, A. F. Parlow, J. Risteli, L. Risteli, Y. C. Lee, T. Feizi, H. Langen, and M. C. Nussenzweig. 2002. Mannose receptor-mediated regulation of serum glycoprotein homeostasis. Science 295:1898-1901.

11. Maeda, N., J. Nigou, J. L. Herrmann, M. Jackson, A. Amara, P. H. Lagrange, G. Puzo, B. Gicquel, and O. Neyrolles. 2003. The cell surface receptor DC-SIGN discriminates between Mycobacterium species through selective recognition of the mannose caps on lipoarabinomannan. J Biol Chem 278:5513-5516.

12. Tailleux, L., O. Schwartz, J. L. Herrmann, E. Pivert, M. Jackson, A. Amara, L. Legres, D. Dreher, L. P. Nicod, J. C. Gluckman, P. H. Lagrange, B. Gicquel, and O. Neyrolles. 2003. DC-SIGN is the major Mycobacterium tuberculosis receptor on human dendritic cells. J Exp Med 197:121-127.

13. Geijtenbeek, T. B., S. J. Van Vliet, E. A. Koppel, M. Sanchez-Hernandez, C. M. Vandenbroucke-Grauls, B. Appelmelk, and Y. Van Kooyk. 2003. Mycobacteria target DC-SIGN to suppress dendritic cell function. J Exp Med 197:7-17.

14. Cooper, A. M., A. Kipnis, J. Turner, J. Magram, J. Ferrante, and I. M. Orme. 2002. Mice lacking bioactive IL-12 can generate protective, antigen-specific cellular responses to mycobacterial infection only if the IL-12 p40 subunit is present. J Immunol 168:1322-1327.

15. Bates, E. E., N. Fournier, E. Garcia, J. Valladeau, I. Durand, J. J. Pin, S. M. Zurawski, S. Patel, J. S. Abrams, S. Lebecque, P. Garrone, and S. Saeland. 1999. APCs express DCIR, a novel C-type lectin surface receptor containing an immunoreceptor tyrosine-based inhibitory motif. J Immunol 163:1973-1983.

16. Bendtsen, J. D., H. Nielsen, G. von Heijne, and S. Brunak. 2004. Improved prediction of signal peptides: SignalP 3.0. J Mol Biol 340:783-795.

17. Shulman, M., C. D. Wilde, G. Kohler, M. J. Shulman, N. S. Rees, D. Atefi, J. T. Homey, S. B. Eaton, W. Whaley, J. T. Galambos, H. Hengartner, L. R. Shapiro, and L. Zemek. 1978.

18. Banchereau, J., F. Briere, C. Caux, J. Davoust, S. Lebecque, Y. J. Liu, B. Pulendran, and K. Palucka. 2000. Immunobiology of dendritic cells. Annu Rev Immunol 18:767-811.

19. Jeannin, P., B. Bottazzi, M. Sironi, A. Doni, M. Rusnati, M. Presta, V. Maina, G. Magistrelli, J. F. Haeuw, G. Hoeffel, N. Thieblemont, N. Corvaia, C. Garlanda, Y. Delneste, and A. Mantovani. 2005. Complexity and complementarity of outer membrane protein A recognition by cellular and humoral innate immunity receptors. Immunity 22:551-560.

20. Wykes, M., and G. MacPherson. 2000. Dendritic cell-B-cell interaction: dendritic cells provide B cells with CD40-independent proliferation signals and CD40-dependent survival signals. Immunology 100: 1-3.

21. Balazs, M., F. Martin, T. Zhou, and J. Kearney. 2002. Blood dendritic cells interact with splenic marginal zone B cells to initiate T-independent immune responses. Immunity 17:341-352.

22. Kikuchi, T., S. Worgall, R. Singh, M. A. Moore, and R. G. Crystal. 2000. Dendritic cells genetically modified to express CD40 ligand and pulsed with antigen can initiate antigen-specific humoral immunity independent of CD4+T cells. Nat Med 6:1154-1159.

23. Dubois, B., J. M. Bridon, J. Fayette, C. Barthelemy, J. Banchereau, C. Caux, and F. Briere. 1999. Dendritic cells directly modulate B cell growth and differentiation. J Leukoc Biol 66:224-230.

24. Qi, H., J. G. Egen, A. Y. Huang, and R. N. Germain. 2006. Extrafollicular activation of lymph node B cells by antigen-bearing dendritic cells. Science 312:1672-1676.

25. Bergtold, A., D. D. Desai, A. Gavhane, and R. Clynes. 2005. Cell surface recycling of internalized antigen permits dendritic cell priming of B cells. Immunity 23:503-514.

26. Ruprecht, C. R., and A. Lanzavecchia. 2006. Toll-like receptor stimulation as a third signal required for activation of human naive B cells. Eur J Immunol 36:810-816.

27. Bernasconi, N. L., E. Traggiai, and A. Lanzavecchia. 2002. Maintenance of serological memory by polyclonal activation of human memory B cells. Science 298:2199-2202.

28. Moore, P. A., O. Belvedere, A. Orr, K. Pieri, D. W. LaFleur, P. Feng, D. Soppet, M. Charters, R. Gentz, D. Parmelee, Y. Li, O. Galperina, J. Giri, V. Roschke, B. Nardelli, J. Carrell, S. Sosnovtseva, W. Greenfield, S. M. Ruben, H. S. Olsen, J. Fikes, and D. M. Hilbert. 1999. BLyS: member of the tumor necrosis factor family and B lymphocyte stimulator. Science 285:260-263.

29. Gross, J. A., J. Johnston, S. Mudri, R. Enselman, S. R. Dillon, K. Madden, W. Xu, J. Parrish-Novak, D. Foster, C. Lofton-Day, M. Moore, A. Littau, A. Grossman, H. Haugen, K. Foley, H. Blumberg, K. Harrison, W. Kindsvogel, and C. H. Clegg. 2000. TACI and BCMA are receptors for a TNF homologue implicated in B-cell autoimmune disease. Nature 404:995-999.

30. Craxton, A., D. Magaletti, E. J. Ryan, and E. A. Clark. 2003. Macrophage- and dendritic cell--dependent regulation of human B-cell proliferation requires the TNF family ligand BAFF. Blood 101:4464-4471.

31. MacLennan, I., and C. Vinuesa. 2002. Dendritic cells, BAFF, and APRIL: innate players in adaptive antibody responses. Immunity 17:235-238.

What is claimed is:

1. A method for increasing the effectiveness of antigen presentation by a CLEC-6-expressing antigen presenting cell comprising contacting the antigen presenting cell with an anti-CLEC-6-specific antibody or fragment thereof, wherein the antigen presenting cell is activated.

2. The method of claim 1, wherein the antigen presenting cell comprises an isolated dendritic cell, a peripheral blood mononuclear cell, a monocyte, a myeloid dendritic cell and combinations thereof.

3. The method of claim 1, wherein the antigen presenting cell comprises an isolated dendritic cell, a peripheral blood mononuclear cell, a monocyte, a B cell, a myeloid dendritic cell and combinations thereof that have been cultured in vitro with GM-CSF and IL-4, interferon alpha, antigen and combinations thereof.

4. The method of claim 1, further comprising the step of activating the antigen presenting cells with GM-CSF and IL-4, wherein contact with the CLEC-6-specific antibody or fragment thereof increases the surface expression of CD86 and HLA-DR on the antigen presenting cell.

5. The method of claim 1, further comprising the step of activating the antigen presenting cells with the CLEC-6-specific antibody or fragment thereof which increases the surface expression of CD86, CD80, and HLA-DR on the antigen presenting cell.

6. The method of claim 1, wherein the antigen presenting cells are dendritic cells that are activated with the CLEC-6-specific antibody and GM-CSF and IL-4 to have the gene expression pattern of FIG. 4.

7. The method of claim 1, wherein the antigen presenting cells are activated with a CLEC-6-specific antibody to secrete IL-6, MIP- 1 a, MCP-1, IP-10, TNFa and combinations thereof.

8. The method of claim 1, wherein the antigen presenting cells are dendritic cells activated with a CLEC-6-specific antibody to secrete IL-6, MIP- 1 a, MCP-1, IP-10, TNFa, IL-12p40, IL- 1 a, IL- 1 b and combinations thereof.

9 . The method of claim 1, wherein the antigen presenting cells comprises a dendritic cell that has been contacted with GM-CSF and IL-4 or Interferon alpha, the CLEC-6-specific antibody or fragment thereof and the CD40 ligand to increase the activation of the dendritic cells.

10. The method of claim 1, wherein the antigen presenting cells comprises a dendritic cell that has been contacted with GM-CSF and IL-4 or Interferon alpha and the CLEC-6-specific antibody or fragment thereof has increased co-stimulatory activity of dendritic cells.

11. The method of claim 1, further comprising the step of co-activating the antigen presenting cell the activating through the TLR9 receptor, wherein the cells increase cytokine and chemokine production.

12. The method of claim 1, further comprising the step of co-activating the antigen presenting cell by activating the cells with GM-CSF, IL-4 and a TLR9 receptor ligand, wherein the dendritic cells trigger B cells proliferation.

13. The method of claim 1, further comprising the step of co-activating the antigen presenting cell by activating the cells with CLEC-6 and LOX- 1 in the presence of B cells, wherein the antigen presenting cells induce B cell immunoglobulin class-switching.

14. The method of claim 1, further comprising the step of co-activating the antigen presenting cell the activating through the TLR9 receptor using at least one of a TLR9 ligand, an anti-TLR9 antibody of fragments thereof, an anti-TLR9-anti-CLEC-6 hybrid antibody or fragment thereof, an anti-TLR9-anti-CLEC-6 ligand conjugate.

15. The method of claim 1, wherein CLEC-6-specific antibody or fragment thereof is selected from clone 12H7, 12E3, 9D5, 20H8 and combinations thereof.

16. The method of claim 1, wherein dendritic cells activated through the CLEC-6-receptor with the CLEC-6-specific antibody or fragment thereof activates monocytes, dendritic cells, peripheral blood mononuclear cells, B cells and combinations thereof.

17. The method of claim 1, wherein CLEC-6-specific antibody or fragment thereof is bound to one half of a Cohesin/Dockerin pair.

18. The method of claim 1, wherein CLEC-6-specific antibody or fragment thereof is bound to one half of a Cohesin/Dockerin pair and the complementary half is bound to an antigen.

19. The method of claim 1, wherein CLEC-6-specific antibody or fragment thereof is bound to one half of a Cohesin/Dockerin pair and the complementary half is bound to an antigen selected from a molecule, a peptide, a protein, a nucleic acid, a carbohydrate, a lipid, a cell, a virus or portion thereof, a bacteria or portion thereof, a fungi or portion thereof, a parasite or portion thereof.

20. The method of claim 1, wherein CLEC-6-specific antibody or fragment thereof is bound to one half of a Cohesin/Dockerin pair and the other half of the pair is bound to one or more cytokines selected from interleukins, transforming growth factors (TGFs), fibroblast growth factors (FGFs), platelet derived growth factors (PDGFs), epidermal growth factors (EGFs), connective tissue activated peptides (CTAPs), osteogenic factors, and biologically active analogs, fragments, and derivatives of such growth factors, B/T-cell differentiation factors, B/T-cell growth factors, mitogenic cytokines, chemotactic cytokines and chemokines, colony stimulating factors, angiogenesis factors, IFN-α, IFN-β, IFN-γ, IL1, IL2, IL3, IL4, IL5, IL6, IL7, IL8, IL9, IL10, IL11, IL12, IL13, IL14, IL15, IL16, IL17, IL18, etc., leptin, myostatin, macrophage stimulating protein, platelet-derived growth factor, TNF-α, TNF-β, NGF, CD40L, CD137L/4- 1 BBL, human lymphotoxin-β, G-CSF, M-CSF, GM-CSF, PDGF, IL-1α, IL1-β, IP-10, PF4, GRO, 9E3, erythropoietin, endostatin, angiostatin, VEGF, transforming growth factor (TGF) supergene family include the beta transforming growth factors (for example TGF- 1 , TGF-2, TGF- 3); bone morphogenetic proteins (for example, BMP-1, BMP-2, BMP-3, BMP-4, BMP-5, BMP-6, BMP-7, BMP-8, BMP-9); heparin-binding growth factors (fibroblast growth factor (FGF), epidermal growth factor (EGF), platelet-derived growth factor (PDGF), insulin-like growth factor (IGF)); Inhibins (for example, Inhibin A, Inhibin B); growth differentiating factors (for example, GDF-1); and Activins (for example, Activin A, Activin B, Activin AB).

21. A method for separating myeloid dendritic cells from plasmacytoid dendritic cells comprising using CLEC-6 expression to isolate myeloid dendritic cells, B cells or monocytes that express CLEC-6 from plasmacytoid dendritic cells which do not express CLEC-6.

22. A hybridoma that expressed a CLEC-6-specific antibody or fragment thereof, wherein the CLEC-6-specific antibody or fragment thereof activates an antigen presenting cell to express new surface markers, secrete one or more cytokines or both.

23. The hybridoma of claim 22, wherein the hybridoma is selected from clone 12H7, 12E3, 9D5, 20H8 and combinations thereof.

24. A method for enhancing B cell immune responses comprising triggering a CLEC-6 receptor on a B cell to increase antibody production, secrete cytokines, increase B cell activation surface marker expression and combinations thereof.

25. The method of claim 24, wherein the B cells secrete IL-8, MIP- 1 a and combinations thereof.

26. The method of claim 24, wherein the B cell increases production of IgM, IgG and IgA.

27. A method for enhancing T cell activation comprising triggering a CLEC-6 receptor on a dendritic cell with a CLEC-6 specific antibody or fragment and contacting a T cell to the CLEC-6 activated dendritic cell, wherein T cell activation is enhanced.

28. The method of claim 27, wherein the T cell is a naive CD8+T cell.

29. The method of claim 27, wherein the dendritic cells are further contacted with GM-CSF and IL-4, interferon alpha, antigen and combinations thereof.

30. The method of claim 27, wherein the T cell increases the secretion of IL-10, IL-15.

31. The method of claim 27, wherein the T cell increases surface expression of 4- 1BBL.

32. The method of claim 27, wherein the T cells proliferate upon exposure to dendritic cells activated with anti-CLEC-6 antibodies or fragments thereof.

33. An anti-CLEC-6 immunoglobulin or portion thereof that is secreted from mammalian cells and an antigen bound to the immunoglobulin.

34. The immunoglobulin of claim 33, wherein the antigen specific domain comprises a full length antibody, an antibody variable region domain, an Fab fragment, a Fab' fragment, an F(ab)₂ fragment, and Fv fragment, and Fabc fragment and/or a Fab fragment with portions of the Fc domain.

35. A vaccine comprising a dendritic cell activated with a CLEC-6-specific antibody or fragment thereof.

36. The vaccine of claim 35, wherein the vaccine is selected from SEQ ID NOS.: 1-7.

37. A modular rAb carrier comprising a CLEC-6-specific antibody binding domain linked to one or more antigen carrier domains that comprise one half of a cohesin-dockerin binding pair.

38. The rAb of claim 37, wherein the antigen-specific binding domain comprises at least a portion of an antibody.

39. The rAb of claim 37, wherein the antigen-specific binding domain comprises at least a portion of an antibody in a fusion protein with the one half of the cohesin-dockerin binding pair.

40. The rAb of claim 37, further comprising a complementary half of the cohesin-dockerin binding pair bound to an antigen that forms a complex with the modular rAb carrier.

41. The rAb of claim 37, further comprising a complementary half of the cohesin-dockerin binding pair that is a fusion protein with an antigen.

42. The rAb of claim 37, wherein the antigen specific domain comprises a full length antibody, an antibody variable region domain, an Fab fragment, a Fab' fragment, an F(ab)2 fragment, and Fv fragment, and Fabc fragment and/or a Fab fragment with portions of the Fc domain.
